(19) United States
(12) Patent Application Publication
(10) Pub. No.: US 2021/0238236 A1
(43) Pub. Date: Aug. 5, 2021

(54) FUSION PEPTIDES OR PROTEINS, THEIR USE, AND SYSTEMS AND KITS BASED THEREUPON, FOR THE SEPARATION AND/OR DETECTION OF PLASTICS, PARTICULARLY OF MICROPLASTICS

(71) Applicant: DWI-LEIBNIZ-INSTITUT FÜR INTERAKTIVE MATERIALIEN E.V., Aachen (DE)

(72) Inventors: Ulrich SCHWANEBERG, Kelmis-Hergenrath (BE); Nursen SÖZER, Übach-Palenberg (DE); Alan MERTENS, Eupen (BE); Mehdi DAVARI DOLATABADI, Aachen (DE); Felix JAKOB, Erkelenz (DE); Shohana ISLAM, Aachen (DE); Lina APITIUS, Dreieich (DE)

(21) Appl. No.: 17/253,291

(22) PCT Filed: Jun. 21, 2019
(86) PCT No.: PCT/EP2019/066535

§ 371 Date: Dec. 17, 2020

(30) Foreign Application Priority Data
Jun. 20, 2018 (EP) 18178726.8

(51) Int. Cl. C07K 14/435; C07K 14/31; C12Q 1/42; G01N 33/44

(52) U.S. Cl. CPC C07K 14/43563; C07K 14/31; C07K 2319/60; C12Q 1/42; G01N 33/442; C07K 14/43509

(57) ABSTRACT

The present invention pertains to a novel fusion protein and/or fusion peptide, preferably for use in the separation from and/or detection in an environment of one or more target polymers or plastics, e.g., one or more target polymer fragments and/M or particles or target plastic fragments and/or particles, preferably wherein the one or more target polymer particles or target plastic particles are microplastics; a method of preparing such novel fusion protein and/or fusion peptide, a system and kit comprising the novel fusion protein and/or fusion peptide and a (polymer or non-polymer) carrier or carrier system, a use of the novel fusion protein and/or fusion peptide or of a system and kit as mentioned in the separation from and/or detection in an environment of one or more target polymers or plastics, e.g., one or more target polymer fragments and/or particles or target plastic fragments and/or particles, preferably wherein the one or more target polymer particles or target plastic particles are microplastics; a method of separation of one or more target polymers or plastics from an environment, e.g., one or more target polymer fragments and/or particles or target plastic fragments and/or particles, and a method of detection of one or more target polymers or plastics in an environment, e.g., one or more target polymer fragments and/or particles or target plastic fragments and/or particles, preferably wherein the one or more target polymer particles or target plastic particles are microplastics.

The present invention pertains to a novel fusion protein and/or fusion peptide, preferably for use in the separation from and/or detection in an environment of one or more target polymers or plastics, e.g., one or more target polymer fragments and/or particles or target plastic fragments and/or particles, preferably wherein the one or more target polymer particles or target plastic particles are microplastics; a method of preparing such novel fusion protein and/or fusion peptide, a system and kit comprising the novel fusion protein and/or fusion peptide and a polymer or non-polymer carrier or carrier system, a use of the novel fusion protein and/or fusion peptide or of a system and kit as mentioned in the separation from and/or detection in an environment of one or more target polymers or plastics, e.g., one or more target polymer fragments and/or particles or target plastic fragments and/or particles, preferably wherein the one or more target polymer particles or target plastic particles are microplastics; a method of separation of one or more target polymers or plastics from an environment, e.g., one or more target polymer fragments and/or particles or target plastic fragments and/or particles, and a method of detection of one or more target polymers or plastics in an environment, e.g., one or more target polymer fragments and/or particles or target plastic fragments and/or particles, preferably wherein the one or more target polymer particles or target plastic particles are microplastics.

Plastic waste in the oceans is a worldwide problem. According to a study published in the scientific journal Science at the beginning of 2015, about 8 million tons of this waste were released into the oceans in 2010, with a confidence interval of 4.8 to 12.7 million tons per year was specified.

Plastic parts, “primary” microplastics as well as the corresponding decomposition products (“secondary” microplastics) accumulate in particular in some ocean drift current vortices and lead to a considerable compression in some marine regions; the North Pacific Gyre brought this phenomenon the nickname Great Pacific Garbage Patch (first described in 1997).

In the oceans driving plastic waste is shredded by wave motion and UV light in the long term, with a higher and higher degree of fineness can be achieved up to the pulverization. At a high degree of fineness, the plastic powder is taken up by various marine inhabitants as well as, among others, plankton instead of or with the usual food. Beginning with plankton, the plastic particles, which may also adhere to toxic and cancer-causing chemicals such as DDT and polychlorinated biphenyls, continue to rise in the food chain. In this way, the plastic waste with the accumulating toxins also reaches the food intended for human consumption. In 2012, the scientific journal Environmental Science & Technology reported on a study on many beaches on all six continents, revealing microplastic particles everywhere.

Microplastics are small plastic particles in the environment. While there is some contention over their size, the U.S. National Oceanic & Atmospheric Administration classifies microplastics as less than 5 mm in diameter. They come from a variety of sources, including cosmetics, clothing, and industrial processes. This includes fibers from fleece and other garments made of synthetic materials: In the waste water of washing machines up to 1900 smallest plastic particles per wash cycle were found.

Two classifications of microplastics currently exist: primary microplastics are manufactured and are a direct result of human material and product use, and secondary microplastics are microscopic plastic fragments derived from the breakdown of larger plastic debris like the macroscopic parts that make up the bulk of the Great Pacific Garbage Patch. Both types are recognized to persist in the environment at high levels, particularly in aquatic and marine ecosystems. The plastic resin beads created for use by manufactures are often called nurdles.

Because plastics do not break down for many years, they can be ingested and incorporated into and accumulated in the bodies and tissues of many organisms. The entire cycle and movement of microplastics in the environment is not yet fully known, but research is currently underway to further investigate this issue.

It is estimated that approximately 5,500-14,000 t of microplastic (plastic particles and fibers smaller than 5 mm) are released into the environment each year, for example into in waters. Microplastics (MP) are either obtained by direct entry into the environment (primary MP) or by fragmentation of larger plastic parts as a result of, for example, mechanical comminution or degradation by UV radiation (secondary MP). MP is considered to be environmentally problematic because it is degraded as plastic only slowly, accordingly it is considered to be persistent and for that reason and because of its small size it gets into the food chain. It is particularly problematical that a large part of the world-wide wastewater reaches the waters as untreated sewage water, and that despite the use of sewage treatment plants many sewage treatment plants remove the MP only insufficiently by conventional filter systems. It should be noted that, in particular of MP of polymers of low density, such as polyethylene (PE, about 27% of world plastics production), polypropylene (PP, about 17% of the world plastics production) and polyurethane foams (PU; about 7% of world plastics production), there is an increased risk potential. This MP of low-density polymers floats in the water or floats on the surface of the water, so it float. floats, and cannot bind to sediment, allowing it to get directly into the food chain of fish, birds, mammals and humans. The direct risk potential for humans based on the plastic microparticles is, according to the experts, rather low. However, the particularly difficult-to-degrade plastics PP and PE often contain plasticizers, which often show hormonal effects, or may contain additives which are carcinogenic, toxic or show endocrine activity. In addition, due to their hydrophobicity, PP and PE bind ubiquitously occurring and highly dangerous contaminants such as PCBs (polychlorinated biphenyls), PAHs (polyaromatic hydrocarbons) or pesticides such as DDT (dichlorodiphenyl trichloroethane).

Information concerning, for example, the degradation of microplastics, the biodegradation of microplastics, the removal of microplastics, the quantification and identification of microplastics, can be found in scientific publications on the state of the art, for example, in the representative scientific publications: Herbort, A. F., Schuhen, K. (2016), “A concept for the removal of microplastics from the marine environment with innovative host-guest relationships”, Environmental Science and Pollution Research, published online: 16 Jul. 2016. DOI 10.1007/s11356-016-7216-x; Ribitsch, D., Herrero Acero, E., Przylucka, A., Zitzenbacher, S., Marold, A., Gamerith, C., Tscheliessnig, R., Jungbauer, A., Rennhofer, H., Lichtenegger, H., Amenitsch, H., Bonazza, K., Kubicek, C. P., Druzhinina, I. S., and Guebitz, G. M. (2015), “Enhanced cutinase-catalyzed hydrolysis of polyethylene terephthalate by covalent fusion to hydrophobins”, Applied Environmental Microbiology, 81:11, p. 3586-92. DOI: 10.1128/AEM.04111-14; Shivan, A. (2011), “New perspectives in plastic biodegradation”, Current Opinion in Biotechnology, 2011, 22: p422-426. DOI 10.1016/j.copbio.2011.01.013; Duis, K. and Coors, A. (2016), “Microplastics in the aquatic and terrestrial environment: sources (with a specific focus on personal care products), fate and effects”, Environmental Sciences Europe, 28:2, 1-25. DOI: 10.1186/s12302-015-0069-y; and Rocha-Santos, T., Duarte, A. C. (2015), “A critical overview of the analytical approaches to the occurrence, the fate and the behavior of microplastics in the environment”, Trends in Analytical Chemistry, 65, p. 47-53. DOI: 10.1016/j.trac.2014.10.011.

A number of disadvantages are evident in the current state of the art, which shall be exemplified as follows.

Since the MP problem is still a young field, there are relatively few approaches to removing MP and the legal regulation (including the assessment of the danger potential) are still rudimentary. Most solutions are not very innovative, cannot distinguish between the microplastic compositions or are not so well developed that they are ready for use. Previous methods for MP detection are also usually consuming, tedious and sometimes faulty.

Although in principle suitable filter systems for sewage and water treatment plants for the separation of microplastics, but these filter systems are not installed in all sewage treatment or water treatment plants or insufficient work (see Fraunhofer UMSICHT “consortium study microplastics/Marine Litter”). For example, an investigation by the Alfred Wegener Institute commissioned by the Oldenburg-East Frisian Water Association found that over 10,000 MP particles per cubic meter of water can be found at the effluent of sewage treatment plants and, depending on the amount of wastewater processed, annual loads of up to more than 14.5 billion MP particles are to be found. In addition, there is no degradation in the filtration and the subsequent disposal of the microplastics (especially in sewage sludge from sewage treatment plants, such as resulting hazardous waste) remains as a question. Furthermore, it should be mentioned that the majority of wastewater is discharged unfiltered into the water world-wide and thus no removal of MP can take place from wastewater.

Activated carbon classic water treatment (often used to filter small amounts of water) is not suitable for removing MP because of the particle size of MP. In addition, there is a risk that adsorbed MP dissolves again and the disposal of contaminated activated carbon is also unclear. Furthermore, activated carbon is expensive and cannot be used on a large scale.

A recently published approach to removing MP from water is based on a container-shaped inclusion compound consisting of a bio-inspired inclusion unit into which the MP is to be captured and finally capped by a capture unit. This concept, which is based on functionalization and sol-gel formation, is so far only of a theoretical nature and remains overall very unconscionable, so that no statement can be made for practical use. Furthermore, here too, only the separation and not the degradation of MP is targeted.

Another concept pursued by the “The Ocean Clean-up” project aims to free oceans of plastic waste. In the process, an artificial coastline is drawn around ocean areas with large amounts of plastic pollution with the help of hose-like shields, the plastic waste is concentrated and then collected. First pilot plants are expected to go into operation in the next few years. The major disadvantage is that practically only gross plastic waste is collected, which floats on the surface. Finer plastic particles or MP particles or fibers, which can swim a little deeper in the water, are not detected. In addition, the concept refers only to large areas of high pollution and the degradation of the plastic is not sought.

The principle possible microbial or enzymatic degradation of plastic and thus microplastics depends strongly on the degradable MP polymer and on other factors such as the presence of suitable microorganisms. While materials such as PET can be degraded (partially) by microorganisms and enzymes (partly as a fusion protein with PET binding hydrophobins), PU by fungi, PVC by bacteria and PE by bacteria and enzymes, the degradation of polypropylene, for example, also remains under optimized laboratory conditions difficult and usually requires a pretreatment with z. B. UV irradiation to allow a subsequent microbial degradation. It should be noted that the microbial and enzymatic degradation of plastic is often possible in principle, but under natural conditions usually abruptly and accordingly runs extremely slowly. Therefore, the microbial or enzymatic degradation so far is not used specifically for the degradation of MP in the environment.

Another problem is the lack of precision in the methods for isolating, detecting, identifying and quantifying MP. For example, so far there is no SOP for the detection of MP. Concerning. The identification and characterization of MP in the laboratory distinguishes between “morphological and physical characterization” and “chemical characterization and quantification”. In the morphological and physical characterization, for example, different screens of different pore size are used and the retained MP particles are examined in each case by optical analyzes such as microscopy. This is tedious, time-consuming and error-prone, since it is sometimes selected with the naked eye. More precise methods are mostly based on SEM (scanning electron microscopy), but require expensive equipment and are usually also expensive. In the chemical characterization and quantification often expensive and complex methods for chemical analysis, such as FT-IR (Fourier transform infrared) or spectroscopy-based methods such as Pyr-GC-MS (pyrolysis-gas chromatography-mass spectrometry) are used. For most of these methods, a complex sample preparation is also necessary.

In view of the worldwide problem of plastic waste, especially of plastic or polymer fragments and/or particles, and specifically of microplastics, in the environment and concerned aqueous media there is a high demand in the state of the art to provide means for the removal from and/or detection in concerned aqueous media, for example, such as any waters, including industrial waters, household waters, waste waters, rivers, lakes, sea, ocean and the like, of plastic or polymer fragments and/or particles, and specifically of microplastics.

Accordingly, it is the object of the present invention to provide efficient means for the separation from and/or detection in an environment, particularly in concerned aqueous media, for example, such as any waters, including industrial waters, household waters, waste waters, rivers, lakes, seas, oceans and the like, of a plastic or polymer material, e.g. one or more target polymers or plastics, particularly one or more target polymer fragments and/or particles or target plastic fragments and/or particles, and specifically wherein the one or more target polymer particles or target plastic particles are microplastics. The term “separation” in the context of the invention denotes the removal of a concerned targeted plastic or polymer material from an environment, but does not include degradation. The term “detection” in the context of the invention denotes the qualitative and/or quantitative analysis of a concerned targeted plastic or polymer material in an environment, and may comprise the removal of, e.g, at least a portion or part of, a concerned targeted plastic or polymer material from an environment, but again does not include degradation.

For solving the object the present invention provides, as defined in the claims and described herein below, a novel fusion protein and/or fusion peptide, preferably for use in the separation from and/or detection in an environment of one or more targeted plastic or polymer materials, e.g. of one or more target polymers or plastics, e.g., one or more target polymer fragments and/or particles or target plastic fragments and/or particles, preferably wherein the one or more target polymer particles or target plastic particles are microplastics; a method of preparing such novel fusion protein and/or fusion peptide, a system and kit comprising the novel fusion protein and/or fusion peptide and a polymer or non-polymer carrier or carrier system, a use of the novel fusion protein and/or fusion peptide or of a system and kit as mentioned in the separation from and/or detection in an environment of one or more target polymers or target plastics, e.g., one or more target polymer fragments and/or particles, preferably wherein the one or more target polymer particles or target plastic particles are microplastics; a method of separation from an environment of one or more target polymers or target plastics, e.g., one or more target polymer fragments and/or particles or target plastic fragments and/or particles, and a method of detection in an environment of one or more target polymers or target plastics, e.g., one or more target polymer fragments and/or particles or target plastic fragments and/or particles, preferably wherein the one or more target polymer particles or target plastic particles are microplastics.

### DESCRIPTION OF FIGURES

FIG. 1: Schematic immobilization of PCL particles (could be microplastic) on stainless steel. Bifunctional fusion proteins immobilize PCL particles on the smooth steel surface. The N-terminal peptide DS1 binds to the stainless steel, whereas the C-terminal LCI binds the PCL particles. The bifunctional fusion protein serves as an adhesion promoter.

FIG. 2: Schematic representation of the protein-based system for removing MP by selective separation of MP consisting of bifunctional fusion proteins in the example set forth, which on the one hand have a function for binding the microplastic surface and on the other hand a function for immobilization on a support. When targeting MP detection, a signal generation function is used instead of a carrier immobilization function.

FIG. 3: Size distribution of PCL particles. The size distribution of the PCL particles was measured by Mie scattering in triplicate. Surface weighted average: 8.66±0.14 μm and volume weighted average: 20.01±0.29 μm. Measuring range 0.020-2000 μM (Micro 15 cc Twin Screw Compounder, Xplore Instruments BV, The Netherlands).

FIG. 4: FESEM analysis of DS1-DZ-LCI mediated immobilization of PCL particles on stainless steel. A) Negative control (PCL particles immobilized on stainless steel with culture supernatant of the pET28 control) at a magnification of 8.4 mm×698. B) PCL particles immobilized on stainless steel with culture supernatant of the fusion protein DS1-DZ-LCI at a magnification of 8.4 mm×700 Images were taken using a field emission scanning electron microscope (S-4800 FE-SEM, Hitachi, Schaumburg, USA). Device settings: Acceleration voltage 3 kV, working distance: 8/8.4 mm, magnification: 70/700×.

FIG. 5: Binding of EGFP-anchor peptide fusion proteins to the analysed polymer materials was determined by confocal fluorescence microscopy. A) PP, PS, and PET as plane surface and B) PP, PS, and PET as microparticles. As negative control EGFP-17H-TEV (without anchor peptide) was used, to determine unspecific binding. Briefly, the negative control displayed no fluorescence on any material under the applied washing conditions. For every tested polymer a suitable anchor peptide for polymer detection was identified.

FIG. 6: The phytase reporter enzyme was immobilized by the anchor peptides CecA, LCI, and TA2 on the target polymers (PS, PP, and PET) and activity was determined using the fluorescent 4-MUP assay. Compared to the phytase wild type all phytase fusion enzymes showed a significantly improved fluorescent signal allowing the detection of microplastic particles.

In a first embodiment the invention relates to a bi- or multifunctional fusion protein and/or fusion peptide, preferably it is directed to a use thereof, comprising
(i) one or more of a first adhesion promoting protein and/or adhesion promoting peptide (I), e.g., an anchor peptide (I), which, preferably selectively, binds to one or more of a first target surface, preferably a first polymer or plastic target surface; and
   preferably wherein the target polymer or target plastic is in the form and/or shape of polymer fragments and/or particles or plastic fragments and/or particles; and
(ii) one or more of a second adhesion promoting protein and/or adhesion promoting peptide (II), e.g., an anchor peptide (II), which, preferably selectively, binds to one or more of a second carrier surface, preferably a second polymer or non-polymer carrier surface;
   and optionally
(iii) a spacer unit between the first and the second adhesion promoting protein and/or adhesion promoting peptide, whereby the first and the second adhesion promoting protein and/or adhesion promoting peptide are bonded together, preferably covalently, by the said spacer unit;
   and/or optionally
(iv) one or more of a function for generating one or more of a signal;
   preferably wherein the bi- or multifunctional fusion protein and/or fusion peptide is for use in the, preferably selective, separation from and/or detection in an environment of one or more target polymers or target plastics, preferably of one or more target polymer particles or target plastic particles.

The use of the before said bi- or multifunctional fusion protein and/or fusion peptide, in the, preferably selective, separation from and/or detection in an environment of one or more target polymers or target plastics, preferably of one or more target polymer particles or target plastic particles, is especially preferred.

Herein, the said detection in an environment of one or more target polymers or target plastics, preferably one or more target polymer particles or target plastic particles, is preferably an identification and/or quantification in an environment of one or more target polymers or target plastics, preferably of one or more target polymer particles or target plastic particles.

In the following, the before said bi- or multifunctional fusion protein and/or fusion peptide itself, the before said use of bi- or multifunctional fusion protein and/or fusion peptide are further described in the context of the present invention.

Herein, the anchor peptide I is an adhesion promoting protein and/or adhesion promoting peptide, which binds, preferably which selectively binds, to one or more of a first target surface, preferably of a first polymer target surface, and is providing for a target binding function, preferably for a selective target binding function.

Herein, the anchor peptide II is an adhesion promoting protein and/or adhesion promoting peptide, which binds, preferably which selectively binds, to one or more of a second carrier surface, preferably of a second polymer or non-polymer carrier surface, and is providing for a carrier binding function, preferably for a selective target binding function.

The anchor peptides can be synthetically or naturally occurring, and typically the anchor peptides (I and/or II) have 2 to 180 amino acids, preferably are derived from natural sources, optionally also modified, for example by means of mutations, for example by typical methods known to the person skilled in the field, e.g. such as one or more point mutations (a genetic mutation where a single nucleotide base is changed, inserted or deleted from a sequence of DNA or RNA) or one or more saturation mutations (a chemo-enzymatic random mutagenesis method applied for the directed evolution of proteins and enzymes; see for example, K. L.; Hauer, B.; Schwaneberg, U. (2005). “Sequence saturation mutagenesis with tunable mutation frequencies”. Anal. Biochem. 341: 187-189. doi:10.1016/j), and/or partially or completely chemically synthesized, for example by typical methods known to the person skilled in the field, e.g. by solid-phase synthesis, e.g., as used in most research and development settings, or by using classical solution-phase synthesis, wherein the solution-phase synthesis techniques have its usefulness, e.g., in large-scale production of peptides for industrial purposes. Peptide coupling agents may be used as known in the technical field, e.g., carbodiimides such as dicyclohexylcarbodiimide (DCC) and diisopropylcarbodiimide (DIC) are frequently used for amide bond formation, as well as protecting group schemes may be applied, as known in the art. When synthesizing medium to long peptides, stepwise elongation techniques may be used, in which the amino acids are connected step-by-step in turn, and which is normally for small peptides containing between 2 and 100 amino acid residues. Another method is fragment condensation, in which peptide fragments are coupled. Still a further method for producing longer peptide chains is chemical ligation, wherein unprotected peptide chains can be reacted chemoselectively in aqueous solution.

The term “carrier” or “carrier surface” denotes a polymer or non-polymer material providing a support function for the anchor peptide II. The carrier or carrier surface may be any material suitable to bind and/or to support, e.g. to immobilize, a protein and/or peptide, for example such carriers and/or supports known to the skilled person also as biocatalyst carriers and/or biocatalyst supports.

The carrier or carrier surface can be or comprise a polymer or non-polymer material. For example, the polymer material can be or comprise any polymer, for example, a polymer selected from the polymers indicated herein also as the target polymers, but it goes without saying that of course the polymer of the carrier or carrier surface is different from the actual target polymer or target plastic. Preferably the carrier or carrier surface can be or comprise a polymer or plastic selected from polystyrene (PS), polypropylene (PP), synthetic fluoropolymer, e.g. synthetic fluoropolymer of tetrafluoroethylene (polytetrafluoroethylene, PTFE), and/or is a polymeric material as used for membranes. The carrier or carrier surface can be or comprise any non-polymer material, for example, a non-polymer material selected from metal, glass, enamel, and ceramic, including metallic, metalized, ceramic, ceramized, glass, glassy, enamel, and enameled materials, woven materials, fiber materials, membrane materials, and combinations thereof. The ceramic and/or ceramized carrier or carrier surface can be or comprise a silica and/or aluminum silicate, The carrier or carrier surface can be or comprise silver (Ag), titanium (Ti), Gold (Au), stainless steel, and/or a magnetic material (e.g. a magnetic particle), a coating (e.g. as a coated woven material, coated fiber materials, and/or coated membrane materials. The carrier or carrier surface can be or comprise a filter and/or filter system.

The carrier or carrier surface can have a variety of different forms and/or shapes. The form and/or shape of the carrier or carrier surface in the context of the invention may widely vary, and includes, for examples, any regular or irregular, spherical or non-spherical, oblong, fibrous, block, powder, granulate, pellet, sphere, filamentous, fibre, film, sheet, mesh, mat, non-woven mat, fabric, scaffold, tube, block, particle, granule and/or three-dimensional construct, and any coexistence thereof and/or combinations thereof.

The carrier or carrier surface can be also a composite, like known for composite catalyst carriers, for example, be a fabric filter or cloth filter or a filtration membrane, or a ceramic or ceramized filter and/or metal filter, or combinations thereof.

It is also possible that the carrier (e.g. a supportive core) and carrier surface thereof (e.g. a coating on the supportive core) are of different materials, e.g., the supportive core of the carrier is made of metal, glass, enamel, and/or ceramic, and the carrier surface (e.g. a coating on the supportive core), is a polymer, for example, as indicated herein also as the target polymers, but it goes without saying that of course the polymer of the carrier surface is different from the target polymer.

The term “spacer unit” denotes a molecule or molecular unit which is or can be inserted between the anchor peptide I and the anchor peptide II, and thereby is linking the anchor peptide I and the anchor peptide II with a (specified) distance. The “spacer unit” can be flexible or rigid and/or stiff, or have any degree of mobility between being flexible or rigid and/or stiff, and/or can be a cleavable linker, and independently the distance provided may be variable, as depending on and chosen by the skilled person according to a selected application, and/or as depending on independently each of the anchor peptide I and the anchor peptide II, or collectively depending on both of the anchor peptide I and the anchor peptide II. The spacer unit may be an inert organic molecule or a peptide and/or protein sequence (e.g. a peptide, or oligopeptide, or polypeptide, respectively, starting from of about four amino acids up to (poly)peptides and/or proteins up to about some hundred amino acids, thus providing a desired distance and a flexible or rigid and/or stiff property, and/or a cleavable linker property, to the bi- or multifunctional fusion protein and/or fusion peptide of the invention, when inserted between and thereby linking, optionally by a cleavable linking, the anchor peptide I and the anchor peptide II.

The “spacer unit” may be of manifold nature, e.g. being any of such known to the skilled person for providing a link and a distance between two proteins and/or peptides. Typical spacer units are, for example, depending on the carrier systems the spacer units can be those known to the skilled person and as disclosed in the state of the art, and for example can comprise or be composed of unstructured and therefore flexible peptide sequences (e.g., as disclosed by: Argos 1990; by Waldo, Standis et al. 1999; or by Klement, Liu et al. 2015); or depending on the carrier systems the spacer units can be those known to the skilled person and as disclosed in the state of the art, and for example can be stiff secondary structure elements of peptides, preferentially stiff helical peptide structures (e.g., as disclosed by: Arai, Ueda et al. 2001; by Amet, Lee et al. 2009; Zhao, Yao et al. 2008); and/or depending on the carrier systems the spacer units can be those known to the skilled person and as disclosed in the state of the art, and for example can be separator proteins, e.g., a Staphylococcal protein A domain Z as disclosed by Tashiro, Tejero et al. 1997. Optionally, alternatively and/or in addition to the before said spacer units, the spacer units can be a cleavable linker known to the skilled person and as disclosed in the state of the art (for example, as disclosed by: Kapust, Toszer et al. 2001; by Chen, Zaro et al. 2013; by Zhao, Xue et al. 2012; or by Schulte 2009). Each of the amino acid sequences indicated herein are coded by the one letter amino acid code.

Preferably the spacer units in general are, for example, selected according to the field of application, e.g. when used in filters or filter systems, as these are described herein below. Preferably the spacer units that are flexible or rigid and/or stiff, or have any degree of mobility between being flexible or rigid and/or stiff, and/or are a cleavable linker, for example, selected according to the field of application, as these are described herein below.

Herein, in particular embodiments the invention also describes novel fusion peptide or fusion protein-based systems and/or kits for the management of microplastics (MP).

The terms “microplastic(s)”, “microplastic particle(s)” or the like denote in the context of the invention plastic(s) or polymer(s), or plastic particle(s) or polymer particle(s), respectively, as a solid material, e.g. particulate material, particularly in partially or completely crystalline form, partially or completely amorphous form, and/or partially or completely glassy form, and/or partially or completely foamed form, as conventionally understood and described by the person skilled in polymer chemistry, having a particle size of less than about 5 mm. Herein the term “particle size” denotes the diameter in spherical, or spherical-like, particles or the length of the longest cross-section of a non-spherical particle. Further Information on the characterization of “microplastic(s)”, “microplastic particle(s)” or the like can be found in a publication of “Landesanstalt für Umwelt, Messungen and Naturschutz Baden-Wurttemberg” related to the characterizing of microplastic, starting at page 20 (see: https://www4.lubw.baden-wuertternberg.de/servlet/is/254486/mikro_kunststoffe.pdf?command=downloadContent&filename=mikro_kunststoffe.pdf).

The form and/or shape of the “microplastic(s)”, “microplastic particle(s)” or the like in the context of the invention plastic(s) or polymer(s), or plastic particle(s) or polymer particle(s), respectively, may widely vary, and includes, for examples, any regular or irregular, spherical or non-spherical, oblong, fibrous, block, powder, granulate, pellet, micropellet, sphere, microsphere, filamentous, fibre and/or microfibre form, and any coexistence thereof and/or combinations thereof. The “microplastic(s)”, “microplastic particle(s)” or the like, as a solid material, e.g. particulate material, can be in a partially or completely crystalline form, partially or completely amorphous form, and/or partially or completely glassy form, and/or partially or completely foamed form, as conventionally understood and described by the person skilled in polymer chemistry.

Herein the terms “plastic” or “polymer”, and the same applies to the terms “copolymer” or “coplastic”, are interchangeable in the context of the invention, with common meaning as normally understood by those skilled in the art, and thus each denote a plastic or polymeric material of high molecular mass, i.e. plastics are typically organic polymers of high molecular mass, and may contain other substances or additives. They are usually synthetic, and most commonly derived from petrochemicals. Thus, the term “plastic” or “polymer” typically denotes a macromolecule whose structure is composed of multiple repeating units of monomers, from which originates a characteristic of high relative molecular mass and attendant properties. The units composing polymers or plastics derive, actually or conceptually, from molecules of low relative molecular mass, called “monomers”. The polymer, as a solid material, e.g. particulate material, such as e.g. fragments and/or particles, can be in a partially or completely crystalline form, partially or completely amorphous form, and/or partially or completely glassy form, and/or partially or completely foamed form, as conventionally understood and described by the person skilled in polymer chemistry.

In a particular embodiment of the invention, the polymers or plastics are such that which floats or do not sediment in a liquid medium, preferably in an aqueous liquid medium of any type, preferably wherein the aqueous liquid medium is water of any type. It may be the case that the polymer or plastic as such floats and/or does not sediment in the said liquid media, and/or it may be the case that the polymer or plastic as such floats and/or does not sediment in the said liquid media while being in the form of a polymer foam or plastic foam.

Herein the terms “peptide” or “protein” are used in the context of the invention with common meaning as normally understood by those skilled in the art, and thus each denote the following. Thus, the terms “peptide” or “protein” denote in particular the following.

Peptides are short chains of amino acid monomers covalently linked by peptide (i.e. amide) bonds. The shortest peptides are dipeptides, consisting of two amino acids joined by a single peptide bond, followed by tripeptides (three amino acids), tetrapeptides (four amino acids), etc. An oligopeptide, often just called peptide, consists of two to twenty amino acids, and thus include dipeptides, tripeptides, tetrapeptides, and pentapeptides, etc. A polypeptide is a long, continuous, and unbranched peptide chain, of from more than twenty amino acids to up approximately 50 amino acids.

Hence, peptides, including oligopeptides and polypeptides, are distinguished from proteins on the basis of size, and as a benchmark can be understood to contain up to approximately 50 amino acids.

A “protein” consists usually of >100 amino acids and can be composed of one or more polypeptides, possibly arranged in a biologically functional way. While aspects of the lab techniques applied to peptides versus polypeptides and proteins differ (e.g., the specifics of electrophoresis, chromatography, etc.), the size boundaries that distinguish peptides from polypeptides and proteins are not absolute in the skilled persons understanding, for example: long peptides such as amyloid beta have been referred to as proteins, and smaller proteins like insulin have been referred to as peptides. Proteins are large biomolecules, or macromolecules, consisting of one or more long chains of amino acid residues. Proteins perform a vast array of functions. Proteins differ from one another primarily in their sequence of amino acids, which is dictated by the nucleotide sequence of their genes, and which usually results in protein folding into a specific three-dimensional structure that determines its activity.

A linear chain of amino acids is called a polypeptide. A protein contains at least one long polypeptide. Short polypeptides, containing less than 20-30 residues, are rarely considered to be proteins and are commonly called peptides, or sometimes oligopeptides. The individual amino acid residues are bonded together by peptide bonds and adjacent amino acid residues. The sequence of amino acid residues in a protein is defined by the sequence of a gene, which is encoded in the genetic code.

Herein the terms “fusion peptide” or “fusion protein” are used in the context of the invention, with common meaning as normally understood by those skilled in the art, and thus each denote the following.

A “fusion protein” and/or “fusion peptide” consists, e.g., are fused together, of one or more proteins and/or of one or more polypeptides, possibly arranged in a biologically functional way. Thus, the terms “fusion protein” and/or “fusion peptide” usually designate hybrid proteins or hybrid peptides, respectively, made of polypeptides having different functions and/or physico-chemical patterns. A “fusion protein” and/or a “fusion peptide” are proteins or peptides, respectively, is normally created through the joining of two or more genes that originally encoded for separate proteins or peptides, respectively. Translation of such fusion gene results in a single or multiple proteins and/or polypeptides with functional properties derived from each of the original proteins or peptides, respectively. Recombinant fusion proteins can be created artificially by recombinant DNA technology. Alternatively fusion proteins can be generated by enzymatically fusing peptides with enzymes, for instances sortases or chemically (e.g. through click-chemistry). Sortases are described, for example as sortase transpeptidases (Structural Biology and Catalytic Mechanism), by Alex W. Jacobitz, Michele D. Kattke, Jeff Wereszczynski, and Robert T. Clubb, in Adv Protein Chem Struct Biol. 2017; 109: 223-264. doi:10.1016/bs.apcsb.2017.04.008.

The invention relating to a bi- or multifunctional fusion protein and/or fusion peptide, preferably for use in the, preferably selective, separation and/or detection, preferably in terms of identification and/or quantification, of one or more target polymers, e.g., fragments and/or particles thereof, including the peptide- and/or protein-based system or kit for the separation and/or detection of MP consists of bi- or multi-functional fusion proteins and/or peptides, on the one hand having at least one function for binding, e.g., the microplastic surface (adhesion promoter protein or peptide), and on the other hand having at least one function for, e.g., attachment to a carrier or carrier system, and optionally having at least one signal generating function. The carrier or carrier system (e.g. filters, membranes, pellets or particles) is characterized by easily being separable from a fluid medium and/or is part of a filter system. The signal generation function(s) may, for example, comprise or consist of a protein or peptide sequence which is, for example, detectable by fluorescence or which binds a dye.

Accordingly, the invention based on a bi- or multifunctional fusion protein and/or fusion peptide, or the use thereof, which is comprising or consisting at least of
(i) one or more of a first adhesion promoting protein and/or adhesion promoting peptide (I), e.g., anchor peptide (I), which, preferably selectively, binds to one or more of a first target surface, preferably of a first polymer or plastic target surface; and
(ii) one or more of a second adhesion promoting protein and/or adhesion promoting peptide (II), e.g., anchor peptide (II), which, preferably selectively, binds to one or more of a second carrier surface, preferably of a second polymer or non-polymer carrier surface.

Furthermore, the invention based on a bi- or multifunctional fusion protein and/or fusion peptide, or the use thereof, is comprising or consisting of at least the “target binding function”/“anchor peptide I” and “carrier binding function”/“anchor peptide II”, may additionally comprise, optionally
(iii) a spacer unit between the first (anchor peptide I) and the second (anchor peptide II) adhesion promoting protein and/or adhesion promoting peptide, whereby the first and the second adhesion promoting protein and/or adhesion promoting peptide are bonded together, preferably covalently, by the said spacer unit;

furthermore, the invention based on a bi- or multifunctional fusion protein and/or fusion peptide, comprising or consisting of at least the “target binding function”/“anchor peptide I” and “carrier binding function”/“anchor peptide II”, may additionally comprise, optionally
(iv) one or more of a function for generating one or more of a signal.

The one or more of a function for generating one or more of a signal can be any type of means, e.g. those known to the skilled person, for a chromogenic and/or fluorometric detection, e.g., through antibodies and/or reporter proteins.

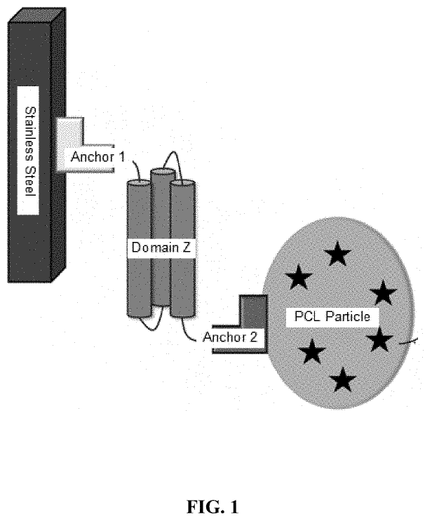

A guide to choosing fluorescent proteins is disclosed by Shaner et al. (2005) in Nature Methods, Vol. 2 No. 12, December 2015, page 905 pp, including the Supplementary FIG. 1, the Supplementary Table 1 and the fluorescent proteins (FPs) not included in main table, the Supplementary Table 2 (pertaining to GFP variants and mutations relative to wtGFP (wild-type GFP), the Supplementary Table 3 (pertaining to photoactivatable and photoconvertible proteins, and the Supplementary Discussion, each as disclosed therein (http://tsienlab.ucsd.edu/Pu blications/Shaner %202005%20Nature %20Methods %20-%20Choosing%20fluorescent%20proteins.pdf).

For example, the reporter protein can be any fluorescent reporter protein, and variants thereof, known in the state of the art. Examples of a fluorescent reporter protein are (enhanced) green fluorescing protein (eGFP) as described by Cormack, Valdivia et al. 1996, Shaner, Steinbach et al. 2005); U.S. Pat. No. 6,172,188); fluorescent reporter proteins described by Tsien (1998) such as yellow fluorescent proteins, cyan fluorescent proteins, blue fluorescent proteins, or mCherry; Cerulan as described by Rizzo, Springer et al. (2004); T-Sapphire as described by Griesbeck, Baird et al. (2001); small ultra-red fluorescent protein (smURFP) as described by Rodriguez, Tran et al. (2016); light oxygen voltage (LOV) based fluorescent proteins, and/or variants thereof, known in the state of the art. Further examples of known fluorescent reporter proteins are given further below.

The reporter protein, for example, can be any enzyme with an enzymatic activity that is inert to (e.g. is compatible with and/or does not cleave) the anchor peptide I and to the second anchor peptide II, or eventually, if present, to a spacer peptide and/or protein. An example of such a reporter protein with an enzymatic activity is a phytase, for example, a phytase that is described in the state of the art, wherein for example phytase is immobilized by means of an anchor peptide onto a surface (Parylene C), and wherein the activity is detected by 4-MUP assay (4-methyl-umbelliferylphosphate). More details on the assay can be found the scientific article by Shivange, A., Roccatano, D., Schwaneberg, U. (2015): Iterative key-residues interrogation of a phytase with thermostability increasing substitutions identified in directed evolution. Appl. Microbiol. Biotechnol., 100, 227-242). Further examples of reporter proteins include Peptides, e.g. peptide sequences, such as for example, strep tag or E-tag for epitope or fluorescent protein binding or antibody binding), reporter proteins with a specific enzymatic activity, or reporter proteins having a Cys (—SH) group for specific chemical labeling, for instance with maleimid fluorophores (e.g. thioglow).

The protein-based system according to the invention preferably serves for, preferably selectively, separating and/or detecting microplastics (MP). As mentioned before, microplastics (MP) particles by definition are understood having a particle size preferably of less than about 5 mm. Within the scope of the invention, however, it is understood that, besides in the millimeter range, preferably of less than about 5 mm, the invention also can serve for, preferably selectively, separating and/or detecting target polymer particles having a particle size in the micrometer and/or nanometer range.

Major potential applications of the novel peptide-based or protein-based systems for removing MP are water treatment e.g. in sewage treatment plants, water treatment plants and managed waters (for example fish farming) and the analysis for the detection and quantification of MP particles, e.g. in waters and food (including beer, honey, mussels).

In summary, the present invention aims at removing microplastics by means of a protein-based system by separation from an environment, in particular from waters, and/or to detect and quantify MP, in particular which are particles, e.g. in partially or completely crystalline form, partially or completely amorphous form, and/or partially or completely glassy form, and/or are partially or completely in the form of a foam (e.g. particles derived from a polymer foam). The separation and detection/quantification of MP with the aid of the protein-based systems according to the invention refers to all common MP polymers, particularly to those which float and/or do not sediment in a liquid medium as indicated herein, and in particular in the water, that is to say MP, based on PE, PP and PU, and also others based on MP, for example Polystyrene (PS), polyvinyl chloride (PVC), polycarbonate (PC), polyamide (PA), polyoxymethylene (POM), polymethyl methacrylate (PMMA), polyethylene terephthalate (PET), polybutylene terephthalate (PBT), polytetrafluoroethylene (PTFE), polyhydroxyalkanoates (PHA), Polyhydroxybutyrate (PHB), polyimide (PI), polylactide (PLA), polyvinylidene fluoride (PVDF) and polyether ketones (PEK etc.). Preferably, the separation of MP with the aid of the protein-based systems according to the invention relates to particularly hydrophobic MPs (which e.g. normally are poorly degradable), which float and/or do not sediment in a liquid medium as indicated herein, and in particular in the water, and/or attract/accumulate toxic compounds (for example, such as PCBs), such as MP based on PE, PP and PU; for example being in particle form or in form of a foam.

In a preferred embodiment the invention can relate to a bi- or multifunctional fusion protein and/or fusion peptide according to the above disclosed invention, wherein the one or more of the first adhesion promoting protein and/or adhesion promoting peptide, preferably selectively, binds to one or more of a first polymer target surface or plastic target surface, preferably wherein the polymer or plastic is selected from the group consisting of polyolefin, in particular polyethylene (PE), polypropylene (PP), polyurethane (PU), polystyrene (PS), polyvinyl chloride (PVC), polycarbonate (PC), polyamide (PA), polyoxymethylene (POM), polymethyl methacrylate (PMMA), polyethylene terephthalate (PET), polybutylene terephthalate (PBT), polytetrafluoroethylene (PTFE), polyhydroxyalkanoate (PHA), polyhydroxybutyrate (PHB), polyimide (PI), polylactide (PLA), polyvinylidene fluoride (PVDF) and polyetherketone (PEK etc.), polyamides (PA), and/or polymeric or plastic foams, and copolymers or coplastics thereof;

more preferably wherein the polymer or plastic is selected from the group consisting of polyethylene (PE), polypropylene (PP), polystyrene (PS), polyurethane (PU), and/or polymeric or plastic foams, and copolymers or coplastics thereof.

In a preferred embodiment the invention can relate to a bi- or multifunctional fusion protein and/or fusion peptide according to the above disclosed invention, wherein the one or more of the first adhesion promoting protein and/or adhesion promoting peptide, preferably selectively, binds to one or more of a first target surface that is a surface of a target polymer or of a target plastic, preferably a surface of a target polymer or of a target plastic which is hydrophobic and/or (environmentally) is particularly difficult or hardly to degrade and/or that binds pollutants and/or toxic, cancerogenic, mutagenic, and/or endocrine active substances. Examples of such substances include pesticides, for example such as dichlorodiphenyl trichloroethane (DDT), polychlorinated biphenyls (PCBs), or polycyclic aromatic hydrocarbons (PAHs), persistent organic pollutants (POPs-substances). Copolymers or co-plastics thereof are included as a surface of a target polymer or of a target plastic.

The target polymer or target plastic is preferably in the form of polymer fragments and/or particles or plastic fragments and/or particles. Herein the term “fragment” denotes a localized object to which can be ascribed several physical or chemical properties such as shape, size, volume, density or mass, for example a piece, part, or particle of any regular or irregular form, for example chip, chinky form, polymer or plastic in a form of shard, sliver, splinter, smithereen, scrap, bit, snip, snippet, wisp, or tatter. The form may comprise edges and/or curves, and independently may comprise a variety of aspect ratios (e.g. in a range of about 5:1 to 1:1), and independently may comprise pores and/or open cells and/or closed cells. Particularly, a fragment and/or particle may be of size in a lower cm range (e.g. about 1.5 to 1 cm), preferably in a sub-μm up to mm range, e.g. in a range of about 0.1 μm to 10 mm. The term “particle” denotes a small, i.e. microscopic to macroscopic, localized object to which can be ascribed several physical or chemical properties such as shape, size, volume, density or mass, of any regular or irregular form, particularly wherein the particle may be of size in a range of less than about 10 mm, preferably in a sub-μm up to mm range, e.g. in a range of up to about 5 mm. A particle minimum size may be in a sub-μm up to medium mm range, e.g. in a sub-μm range, e.g. of less than 1 μm, wherein particle sizes are comprised of below 300 nm (below 0.3 μm) that can pass the blood-brain barrier; or wherein particle sizes are comprised of up to about 10 mm, preferably of up to about 5 mm, e.g. in a range of about 0.1 μm to about 5 mm, of about 0.1 μm to about 4 mm, of about 0.1 μm to about 3 mm, of about 0.1 μm to about 2 mm or of about 0.1 μm to about 1 mm, respectively. In an embodiment, a particle is more or less rounded and/or (at least approximately) oblong to spherical (e.g. with an aspect ratio in a range of about 2:1 to 1:1).

In a preferred embodiment the invention can relate to a bi- or multifunctional fusion protein and/or fusion peptide according to the above disclosed invention, wherein the target polymer or target plastic is in the form of polymer fragments and/or particles or plastic fragments and/or particles, preferably in the form and/or shape of polymer and/or plastic microparticles, of polymer and/or plastic microfibers, of polymer and/or plastic microspheres, and/or of polymer and/or plastic micropellets, (commonly “microplastics (MP)”), more preferably in the form and/or shape, e.g. in partially or completely crystalline form, partially or completely amorphous form, and/or partially or completely glassy form, and/or are partially or completely in the form of a foam (e.g. particles derived from a polymer foam), of polymer microparticles (“microplastics (MP)”), preferably which float and/or do not sediment in a liquid medium, preferably in an aqueous liquid medium, preferably wherein the aqueous liquid medium is water, and even more preferably microparticles (“microplastics (MP)”) based on polyethylene (PE), polypropylene (PP), polystyrene (PS), polyurethane (PU), and/or polymeric or plastic foams, and copolymers or coplastics thereof.

In a particular embodiment, the present invention pertains to the use of the bi- or multifunctional fusion protein and/or fusion peptide as described herein, wherein the one or more of the first adhesion promoting protein and/or adhesion promoting peptide binds to one or more of a first target surface that is a surface of a target polymer or of a target plastic, and wherein the bi- or multifunctional fusion protein and/or fusion peptide is for use in the separation from and/or detection in an environment of one or more target polymers or target plastics, wherein the said environment is that of or an environment related to a production, processing, packaging, and/or bottling process and/or quality and/or safety control process of any one of food, beverages, diets, functional food, functional beverages, medical food, pharmaceutical preparations, nutraceuticals, cosmetic preparations, body care preparations, animal feed, including pet feed.

In a further particular embodiment, the present invention pertains to the use of the bi- or multifunctional fusion protein and/or fusion peptide as described herein, wherein the said environment is that of or an environment related to a production, processing, packaging, and/or bottling process and/or quality and/or safety control process of any one of food, beverages, drinking water, diets, functional food, functional beverages; preferably wherein the said environment is that of or an environment related to a production, processing, packaging, and/or bottling process and/or quality and/or safety control process of any one of food, beverages (e.g. beer, wine, fruit juice, lemonade, soft drink) and drinking water.

The bi- or multifunctional fusion protein and/or fusion peptide according to the above disclosed invention, wherein polymer or plastic microparticles (“microplastics (MP)”) have a particle size of less than or equal to about 10 mm, preferably of less or equal to about 5 mm, more preferably of less than about 1 μm, and even more preferably of less than or approximately of about 0.3 μm (300 nm). The particle size can be measured according to methods known to the skilled person, for example by microscopy in the particle range of from about 1 μm and above, or by dynamic light scattering (e.g. Zetasizer) in the particle range of from about 1 μm and below.

In a preferred embodiment the invention can relate to a bi- or multifunctional fusion protein and/or fusion peptide according to the above disclosed invention, wherein the first adhesion promoting protein and/or adhesion promoting peptide, and/or the second adhesion promoting protein and/or adhesion promoting peptide, independently from each other, is selected from the group consisting of anchor peptides (I and/or II) having 2 to 180 amino acids, preferably independently from each other are derived from natural sources and/or chemically synthesized and/or tailored by means of protein engineering;

preferably wherein the first adhesion promoting protein and/or adhesion promoting peptide, and/or the second adhesion promoting protein and/or adhesion promoting peptide, independently from each other, is selected from peptides having 2 to 180 amino acids which have (natural) ability to integrate into membranes of microorganisms, can bind to a (polymer or plastic) target surface (diverse polymer or plastic surfaces).

As described before, the anchor peptides (I and/or II, independently from each other) are characterized by their ability to bind, for example, particularly to polymers, stainless steels, ceramics, etc. Typical examples of such anchor peptides are described, e.g., in the European patent application EP 3261435 A1 directed to plant protection and/or plant growth promotion system.

The adhesion promoting protein and/or adhesion promoting peptide, e.g., the anchor peptide I and/or the anchor peptide II, independently from each other, can be unstructured or linear, or they can comprise or consist of α-helices, β-sheets, and α-helices/β-sheets. Particularly preferred anchor peptides comprise, and more preferably consist of, β-sheets.

Anchor peptides suitable in the context of the invention can be e.g. any of those known to the skilled person. Examples of anchor peptides include Androctonin as described by Ehret-Sabatier, Loew et al. (1996); Antifungal protein 1 as described by Shao, Hu et al. (1999); Cecropin A as described by Steiner, Hultmark et al. (1981); Cg-Def as described by Gueguen, Herpin et al. (2006); Dermaseptin S1 as described by Brand, Leite et al. (2002); hDermcidin as described by Schittek, Hipfel et al. (2001); Liquid Chromatography Peak 1 “LCI” as described by Gong, Wang et al. (2011); Macaque histatin as described by Xu, Telser et al. (1990); MBP-1 as described by Duvick, Rood et al. (1992); Plantaricin A as described by Nissen-Meyer, Larsen et al. (1993); PP102 as described by Shen, Ye et al. (2010); Psoriasin as described by Glaser, Harder et al. (2005); Tachystatin A2 as described by Osaki, Omotezako et al. (1999); and/or Thanatin as described by Fehlbaum, Bulet et al. (1996).

In a preferred embodiment the invention can relate to a bi- or multifunctional fusion protein and/or fusion peptide according to the above disclosed invention, wherein the first adhesion promoting protein and/or adhesion promoting peptide, and/or the second adhesion promoting protein and/or adhesion promoting peptide, independently from each other, is selected from the group consisting of Cecropin A, Tachystatin A2 (TA2), Thanatin (THA), Liquid Chromatography Peak 1 (LCI), Androctonin (ANR), Dermaseptin S1 (DS1), and a combination thereof;
preferably wherein the first adhesion promoting protein and/or adhesion promoting peptide (anchor peptide I) is selected from the group consisting of Tachystatin A2 (TA2), Thanatin (THA), LCI, Dermaseptin S1 (DS1), and a combination thereof;
and/or
preferably wherein the second adhesion promoting protein and/or adhesion promoting peptide (anchor peptide II) is selected from the group consisting of Tachystatin A2 (TA2), Thanatin (THA), Liquid Chromatography Peak 1 (LCI), Dermaseptin S1 (DS1), hDermcidin (hDerm), and a combination thereof;
and
with the proviso that the selected second adhesion promoting protein and/or adhesion promoting peptide (anchor peptide II) is different from the selected first adhesion promoting protein and/or adhesion promoting peptide (anchor peptide I).

In a preferred embodiment the invention can relate to a bi- or multifunctional fusion protein and/or fusion peptide according to the above disclosed invention, wherein the second adhesion promoting protein and/or adhesion promoting peptide, which, preferably selectively, binds to one or more of a second polymer or non-polymer carrier surface, preferably wherein the second polymer or non-polymer carrier surface is selected from the group consisting of metallic, metalized, ceramic, ceramized, glass, glassy, enamel, enamelled materials, woven materials, fiber materials, membrane materials, and a combination thereof; preferably a metallic or metalized material, silver (Ag), titanium (Ti), Gold (Au), stainless steel, and/or a magnetic material (e.g. a magnetic particle); more preferably titanium (Ti), Gold (Au), and/or stainless steel.

In a preferred embodiment the invention can relate to a bi- or multifunctional fusion protein and/or fusion peptide according to the above disclosed invention, wherein the one or more of a function for generating one or more of a signal comprises at least one signal generation function based on a protein sequence which is detectable by fluorescence or which binds a dye or pigment, preferably wherein the one or more of a function for generating one or more of a signal comprises at least one signal generation function based on a protein sequence which is detectable by fluorescence.

In a preferred embodiment the invention can relate to a bi- or multifunctional fusion protein and/or fusion peptide according to the above disclosed invention, wherein the one or more of a function for generating one or more of a signal comprises at least one signal generation function based on a protein sequence which is detectable by fluorescence as described above, e.g. antibodies (such as E-tag epitope) or Strep-Tag with chromeo-labelled streptavidin;

preferably wherein the protein sequence which is detectable by fluorescence is a fluorescent reporter protein, and variants thereof, selected from the group consisting of (enhanced) green fluorescing protein (eGFP), yellow fluorescent proteins, cyan fluorescent proteins, blue fluorescent proteins, mCherry, Cerulan, T-Sapphire, small ultra-red fluorescent protein (smURFP), light oxygen voltage (LOV) based fluorescent proteins; preferably green fluorescing protein (eGFP) and/or variants thereof, light oxygen voltage (LOV) based fluorescent proteins, and/or mCherry.

In a preferred embodiment the invention can relate to a bi- or multifunctional fusion protein and/or fusion peptide according to the above disclosed invention, wherein the protein sequence which is detectable by fluorescence is attached to the C-terminus of first adhesion promoting protein and/or adhesion promoting peptide and/or is attached to the N-terminus of the second adhesion promoting protein and/or adhesion promoting peptide.

In a preferred embodiment the invention can relate to a bi- or multifunctional fusion protein and/or fusion peptide according to the above disclosed invention, wherein (i) the second adhesion promoting protein and/or adhesion promoting peptide is present, and wherein the first adhesion promoting protein and/or adhesion promoting peptide and the second adhesion promoting protein and/or adhesion promoting peptide are bonded together, preferably covalently, by a spacer unit, as defined above, particularly wherein the spacer units are a flexible or rigid and/or stiff, or have any degree of mobility between being flexible or rigid and/or stiff, and/or are a cleavable linker;
preferably wherein the spacer unit comprises or is composed of a unstructured and therefore flexible peptide sequence, or comprises or is composed of a stiff secondary structure element of a peptide, preferentially a stiff helical peptide structure, and/or wherein the spacer unit comprises or is composed of a large spacer unit selected from a polypeptide and/or protein as defined above, preferably wherein the spacer unit a large spacer unit is selected from a separator protein;
and even more preferably wherein the spacer unit is selected from a spacer unit which is a large spacer unit selected from a polypeptide and/or protein, preferably wherein the spacer unit is a separator protein, even more preferably wherein the separator protein is a domain Z separator protein, e.g. separator proteins of a Staphylococcal protein A domain Z;
optionally, alternatively and/or in addition to the before said spacer units, wherein the spacer unit comprises or is composed of a cleavable linker.

In a preferred embodiment the invention can relate to a bi- or multifunctional fusion protein and/or fusion peptide according to any of the before disclosed embodiments of the invention, wherein the second adhesion promoting protein and/or adhesion promoting peptide is N-terminal DS1 (“DS1”=Dermaseptin S1), and wherein the first adhesion promoting protein and/or adhesion promoting peptide is C-terminal Tachystatin A2 (TA2), THA (“THA”=Thanatin), LCI (“LCI”=Liquid Chromaography Peak 1), preferably C-terminal THA (“THA”=Thanatin) or LCI (“LCI”=Liquid Chromaography Peak 1).

In a preferred embodiment the invention can relate to a bi- or multifunctional fusion protein and/or fusion peptide according to any of the before disclosed embodiments of the invention, wherein the spacer unit is a large spacer unit selected from the above named polypeptide and/or protein ones, preferably wherein the spacer unit is a domain Z separator protein, preferably wherein domain Z separator protein is separating the second adhesion promoting protein and/or adhesion promoting peptide represented by N-terminal DS1 (“DS1”=Dermaseptin S1) and the first adhesion promoting protein and/or adhesion promoting peptide represented by C-terminal Tachystatin A2 (TA2), THA (“THA”=Thanatin), LCI (“LCI”=Liquid Chromaography Peak 1), preferably C-terminal THA (“THA”=Thanatin) or LCI (“LCI”=Liquid Chromaography Peak 1).

In a preferred embodiment the invention can relate to a bi- or multifunctional fusion protein and/or fusion peptide according to any of the before disclosed embodiments of the invention, wherein the spacer unit is a large spacer unit selected from the above named polypeptide and/or protein ones, preferably wherein the spacer unit is a domain Z separator protein, preferably wherein the domain Z separator protein is separating the second adhesion promoting protein and/or adhesion promoting peptide represented by N-terminal DS1 (“DS1”=Dermaseptin S1) and the first adhesion promoting protein and/or adhesion promoting peptide represented by C-terminal Tachystatin A2 (TA2), THA (“THA”=Thanatin), LCI (“LCI”=Liquid Chromaography Peak 1), preferably C-terminal THA (“THA”=Thanatin) or LCI (“LCI”=Liquid Chromaography Peak 1), and wherein the protein sequence which is detectable by fluorescence as defined above, preferably wherein the protein sequence is detectable by fluorescence of green fluorescing protein (eGFP) and/or variants thereof, light oxygen voltage (LOV) based fluorescent proteins, and/or mCherry.

The present invention, in one embodiment, also relates to a novel bi- or multifunctional fusion protein and/or fusion peptide, preferably for use in the separation from and/or detection in an environment of one or more target polymers or target plastics, comprising:
(i) one or more of a first adhesion promoting protein and/or adhesion promoting peptide (I), which binds to one or more of a first polymer or plastic target surface;
(ii) one or more of a second adhesion promoting protein and/or adhesion promoting peptide (II), which binds to one or more of a second polymer or non-polymer carrier surface; and
(iii) a spacer unit between the first and the second adhesion promoting protein and/or adhesion promoting peptide, whereby the first and the second adhesion promoting protein and/or adhesion promoting peptide are bonded together, preferably covalently, by the said spacer unit,
   wherein the spacer units are a flexible or rigid and/or stiff, or have any degree of mobility between being flexible or rigid and/or stiff, and/or are a cleavable linker;
   and preferably wherein the spacer unit comprises or is composed of a unstructured and therefore flexible peptide sequence, or comprises or is composed of a stiff secondary structure element of a peptide, preferentially a stiff helical peptide structure, and/or wherein the spacer unit comprises or is composed of a large spacer unit selected from a polypeptide and/or protein as defined above, preferably wherein the spacer unit a large spacer unit is selected from a separator protein;
      and/or optionally
(iv) one or more of a function for generating one or more of a signal.

In said embodiment, the novel bi- or multifunctional fusion protein and/or fusion peptide according to the invention, preferably which is for use in the separation from and/or detection in an environment of one or more target polymers or target plastics, in (iii) the spacer unit is preferably a separator protein.

In said embodiment, the novel bi- or multifunctional fusion protein and/or fusion peptide according to the invention, preferably which is for use in the separation from and/or detection in an environment of one or more target polymers or target plastics, in (iii) the spacer unit is more preferably a domain Z separator protein, most preferably wherein in (iii) the spacer unit is a separator protein of a Staphylococcal protein A domain Z.

In another embodiment the invention also pertains to the before said bi- or multifunctional fusion protein and/or fusion peptide for use in the separation from and/or detection in an environment of one or more target polymers or target plastics. Herein, preferably the said bi- or multifunctional fusion protein and/or fusion peptide for use according to the invention, is such wherein the one or more of the first adhesion promoting protein and/or adhesion promoting peptide binds to one or more of a first target surface that is a surface of a target polymer or of a target plastic, and wherein the bi- or multifunctional fusion protein and/or fusion peptide is for use in the separation from and/or detection in an environment of one or more target polymers or target plastics, wherein the said environment is that of or an environment related to a production, processing, packaging, and/or bottling process and/or quality and/or safety control process of any one of food, beverages, diets, functional food, functional beverages, medical food, pharmaceutical preparations, nutraceuticals, cosmetic preparations, body care preparations, animal feed, including pet feed.

In said another embodiment the invention the bi- or multifunctional fusion protein and/or fusion peptide for use according to the invention, is such wherein the said environment is that of or an environment related to a production, processing, packaging, and/or bottling process and/or quality and/or safety control process of any one of food, beverages, drinking water, diets, functional food, functional beverages; preferably wherein the said environment is that of or an environment related to a production, processing, packaging, and/or bottling process and/or quality and/or safety control process of any one of food, beverages (e.g. beer, wine, fruit juice, lemonade, soft drink) and drinking water.

In another embodiment the invention relates to a system, preferably for use in the separation from and/or in an environment of one or more target polymers or target plastics, preferably fragments and/or particles thereof, comprising:
(A) a bi- or multifunctional fusion protein and/or fusion peptide comprising:
   (i) one or more of a first adhesion promoting protein and/or adhesion promoting peptide (I), which, preferably selectively, binds to one or more of a first polymer or plastic target surface; and
   (ii) one or more of a second adhesion promoting protein and/or adhesion promoting peptide (II), which, preferably selectively, binds to one or more of a second polymer or non-polymer carrier surface;
   and optionally
   (iii) a spacer unit between the first and the second adhesion promoting protein and/or adhesion promoting peptide, whereby the first and the second adhesion promoting protein and/or adhesion promoting peptide are bonded together, preferably covalently, by the said spacer unit;
   and/or optionally
   (iv) one or more of a function for generating one or more of a signal;
      and
(B) one or more of a second polymer or non-polymer carrier surface, which is bonded to the one or more of the second adhesion promoting protein and/or adhesion promoting peptide (II).

In this embodiment of a system the invention also relates to a system, preferably for use in the, preferably selective, separation from and/or detection, preferably in terms of identification and/or quantification, in an environment of one or more target polymers or target plastics, preferably fragments and/or particles thereof, comprising
(A) a bi- or multifunctional fusion protein and/or fusion peptide (I), preferably for use in the separation from and/or detection in an environment of polymer fragments and/or particles or plastic fragments and/or particles, as defined above, and in the claims;
   and/or
(B) a polymer or non-polymer carrier or carrier system that, preferably selectively, binds to the one or more of a second adhesion promoting protein and/or adhesion promoting the peptide (II) of said bi- or multifunctional fusion protein and/or fusion peptide, preferably for use in the separation from and/or detection in an environment of polymer or target plastics particles or plastic fragments and/or particles, as defined above, and in the claims.

In still another embodiment the invention relates to kit, preferably for use in the separation from and/or in an environment of one or more target polymers or target plastics, preferably fragments and/or particles thereof, comprising:
(A) a first component comprising or consisting of a bi- or multifunctional fusion protein and/or fusion peptide comprising:
   (i) one or more of a first adhesion promoting protein and/or adhesion promoting peptide (I), which, preferably selectively, binds to one or more of a first polymer or plastic target surface; and
   (ii) one or more of a second adhesion promoting protein and/or adhesion promoting peptide (II), which, preferably selectively, binds to one or more of a second polymer or non-polymer carrier surface;
   and optionally
   (iii) a spacer unit between the first and the second adhesion promoting protein and/or adhesion promoting peptide, whereby the first and the second adhesion promoting protein and/or adhesion promoting peptide are bonded together, preferably covalently, by the said spacer unit;
   and/or optionally
   (iv) one or more of a function for generating one or more of a signal;
      and
(B) a second component comprising or consisting of one or more of a second polymer or non-polymer carrier surface, which, preferably selectively, binds to the one or more of the second adhesion promoting protein and/or adhesion promoting peptide (II).

In this embodiment of a kit the invention also relates to a kit, preferably for use in the, preferably selective, separation from and/or detection, preferably identification and/or quantification, in an environment of one or more target polymers or target plastics, preferably fragments and/or particles thereof, comprising
(A) a first component comprising or consisting of a bi- or multifunctional fusion protein and/or fusion peptide (I), preferably for use in the separation from and/or detection in an environment of polymer fragments and/or particles or plastic fragments and/or particles, as defined above, and in the claims;
   and
(B) a second component comprising or consisting of a polymer and/or non-polymer carrier or carrier system that, preferably selectively, binds to the one or more of a second adhesion promoting protein and/or adhesion promoting the peptide (II) of said bi- or multifunctional fusion protein and/or fusion peptide, preferably for use in the separation from and/or detection in an environment of polymer fragments and/or particles or plastic fragments and/or particles, as defined above, and in the claims.

In a preferred embodiment the invention can relate to a system as defined above, and in the claims, or a kit as defined above, and in the claims, wherein the carrier or carrier system has a non-polymer carrier surface, preferably wherein the non-polymer carrier surface is selected from the group consisting of metallic, metalized, ceramic, ceramized, glass, glassy, enamel, enameled glassy, enamel, enamelled materials, woven materials, fiber materials, membrane materials, and a combination thereof; preferably a metallic or metalized material, silver (Ag), titanium (Ti), Gold (Au), stainless steel, and/or a magnetic material (e.g. a magnetic particle); more preferably titanium (Ti), Gold (Au), and/or stainless steel.

Particularly, in an embodiment the invention pertains to a system according to the invention, or a kit according to the invention, which is for use in the separation from and/or detection in an environment of one or more target polymers or target plastics.

In said embodiment, the system for use according to the invention and/or kit for use according to the invention, is such wherein the one or more of the first adhesion promoting protein and/or adhesion promoting peptide binds to one or more of a first target surface that is a surface of a target polymer or of a target plastic, and wherein the bi- or multifunctional fusion protein and/or fusion peptide is for use in the separation from and/or detection in an environment of one or more target polymers or target plastics, wherein the said environment is that of or an environment related to a production, processing, packaging, and/or bottling process and/or quality and/or safety control process of any one of food, beverages, diets, functional food, functional beverages, medical food, pharmaceutical preparations, nutraceuticals, cosmetic preparations, body care preparations, animal feed, including pet feed.

In said embodiment, the system for use according to the invention and/or kit for use according to the invention, is such wherein the said environment is that of or an environment related to a production, processing, packaging, and/or bottling process and/or quality and/or safety control process of any one of food, beverages, drinking water, diets, functional food, functional beverages; preferably wherein the said environment is that of or an environment related to a production, processing, packaging, and/or bottling process and/or quality and/or safety control process of any one of food, beverages (e.g. beer, wine, fruit juice, lemonade, soft drink) and drinking water.

The system as defined above, and in the claims, or the kit as defined above, and in the claims, wherein the carrier or carrier system that, preferably selectively, binds to the one or more of a second adhesion promoting protein and/or adhesion promoting peptide which is selected from the group consisting of DS1 (“DS1”=Dermaseptin S1), Cecropin A (CecA), e.g., CecA as disclosed by: Hakan, S., Andreau, D., Merrifield, R. B., 1988. Binding and action of Cecropin and Cecropin analogues: antibacterial peptides from insects. Biochim. Biophys. Acta 939, 260-266), Tachystatin A2 (TA2), LCI (“LCI”=Liquid Chromaography Peak 1), preferably DS1 (“DS1”=Dermaseptin S1), and a combination thereof.

The system as defined above, and in the claims, or the kit as defined above, and in the claims, wherein the carrier or carrier system is in a form that allows for easy separability and/or is part of an adsorber and/or a filter system.

In a further embodiment the invention relates to a use of a bi- or multifunctional fusion protein and/or fusion peptide as defined above, and in the claims, or of a system as defined above, and in the claims, or of a kit as defined above, and in the claims, in the, preferably selective, separation from and/or detection, preferably in terms of identification and/or quantification, in an environment of one or more target polymers or target plastics, preferably fragments and/or particles thereof,
preferably of a target polymer or of a target plastic, preferably a surface of a target polymer or of a target plastic which is hydrophobic and/or (environmentally) is particularly difficult or hardly to degrade and/or that binds pollutants and/or toxic, cancerogenic, mutagenic, and/or endocrine active substances, for example active substances including pesticides, for example such as dichlorodiphenyl trichloroethane (DDT), polychlorinated biphenyls (PCBs), or polycyclic aromatic hydrocarbons (PAHs), persistent organic pollutants (POPs-substances);
more preferably wherein the target polymer or target plastic is in the form and/or shape of polymer fragments and/or particles or plastic fragments and/or particles, preferably in the form and/or shape, e.g. in partially or completely crystalline form, partially or completely amorphous form, and/or partially or completely glassy form, and/or are partially or completely in the form of a foam (e.g. particles derived from a polymer foam), of polymer and/or plastic microparticles (“microplastics (MP)”), more preferably in the form and/or shape of polymer and/or plastic microparticles, of polymer and/or plastic microfibers, of polymer and/or plastic microspheres, and/or of polymer and/or plastic micropellets, (commonly “microplastics (MP)”), preferably which float and/or do not sediment in a liquid medium, preferably in an aqueous liquid medium, preferably wherein the aqueous liquid medium is water, and even more preferably microparticles (“microplastics (MP)”) based on polyethylene (PE), polypropylene (PP), polystyrene (PS), polyurethane (PU), and/or polymeric or plastic foams, and copolymers or coplastics thereof.

The use as defined here before, and in the claims of the bi- or multifunctional fusion protein and/or fusion peptide as defined above and in the claims, or of a system as defined above, and in the claims, or of a kit as defined above, and in the claims, wherein polymer or plastic microparticles (“microplastics (MP)”), e.g. in partially or completely crystalline form, partially or completely amorphous form, and/or partially or completely glassy form, and/or are partially or completely in the form of a foam (e.g. particles derived from a polymer foam), have a particle size of less than or equal to about 10 mm, preferably of less or equal to about 5 mm, more preferably of less than about 1 μm, and even more preferably of less than or approximately of about 0.3 μm (300 nm). The particle size can be measured according to methods known to the skilled person, for example by microscopy in the particle range of from about 1 μm and above, or by dynamic light scattering (e.g. Zetasizer) in the particle range of from about 1 μm and below.

Suitable methods for determining the particle size and/or particle size distribution, recourse can be had to the methods known to the person skilled in the art, e.g. Dynamic Image Analysis (DIA), Static Laser Light Scattering (SLS, also laser diffraction) and Sieve Analysis, are the most common methods for particle size measurement. The sieve analysis is suitable for the measurement in the particle size range of about 20 μm (microns) to about 30 mm. The light scattering, such as the named Static Laser Light Scattering (SLS, also laser diffraction) is suitable for measurements in the particle size range of about 10 nm to about 5 mm.

Suitable methods for determining the particle size and/or particle size distribution, recourse can be had to the methods known to the person skilled in the art, e.g. Dynamic Image Analysis (DIA), Static Laser Light Scattering (SLS, also laser diffraction) and Sieve Analysis, are the most common methods for particle size measurement. The sieve analysis is suitable for the measurement in the particle size range of about 20 μm (microns) to about 30 mm. The light scattering, such as the named Static Laser Light Scattering (SLS, also laser diffraction) is suitable for measurements in the particle size range of about 10 nm to about 5 mm.

The use as defined here before, and in the claims of the bi- or multifunctional fusion protein and/or fusion peptide as defined in the claims, or of a system as defined above, and in the claims, or of a kit as defined above, and in the claims, wherein the polymer or plastic, e.g. in partially or completely crystalline form, partially or completely amorphous form, and/or partially or completely glassy form, and/or are partially or completely in the form of a foam (e.g. particles derived from a polymer foam), is selected from the group consisting of polyolefin, in particular polyethylene (PE), polypropylene (PP), polyurethane (PU), polystyrene (PS), polyvinyl chloride (PVC), polycarbonate (PC), polyamide (PA), polyoxymethylene (POM), polymethyl methacrylate (PMMA), polyethylene terephthalate (PET), polybutylene terephthalate (PBT), polytetrafluoroethylene (PTFE), polyhydroxyalkanoate (PHA), polyhydroxybutyrate (PHB), polyimide (PI), polylactide (PLA), polyvinylidene fluoride (PVDF) and polyetherketone (PEK etc.), and/or polymeric or plastic foams, and copolymers or coplastics thereof; more preferably wherein the polymer or plastic is selected from the group consisting of any of the preferred ones as defined above (e.g. such as PP, PE, PS, PU); and/or a filter system.

In still a further embodiment the invention relates to a use of a bi- or multifunctional fusion protein and/or fusion peptide as defined above, and in the claims, or of a system as defined above, and in the claims, or of a kit as defined above, and in the claims, in separation and/or detection applications, in particular related to an environment of a liquid medium, preferably in an aqueous liquid medium, preferably wherein the aqueous liquid medium is water, in the fields selected from water treatment, sewage treatment plants, water treatment plants, managed waters, and fish farming; analysis, detection and/or quantification, preferably of microplastics and/or MP particles, e.g. in partially or completely crystalline form, partially or completely amorphous form, and/or partially or completely glassy form, and/or are partially or completely in the form of a foam (e.g. particles derived from a polymer foam), in waters, natural waters (e.g., such as rivers, lakes, sea, ice), drinking waters, production waters, food and beverages (e.g., including beer, honey, mussels).

Furthermore, the invention also pertains to a use of a bi- or multifunctional fusion protein and/or fusion peptide as defined in the claims, or of a system as defined in the claims, or of a kit as defined in the claims, in separation and/or detection applications, in particular in the separation from and/or detection in an environment of one or more target polymers or target plastics, wherein the one or more of the first adhesion promoting protein and/or adhesion promoting peptide binds to one or more of a first target surface that is a surface of a target polymer or of a target plastic, and wherein the bi- or multifunctional fusion protein and/or fusion peptide is for use in the separation from and/or detection in an environment of one or more target polymers or target plastics, wherein the said environment is that of or an environment related to a production, processing, packaging, and/or bottling process and/or quality and/or safety control process of any one of food, beverages, diets, functional food, functional beverages, medical food, pharmaceutical preparations, nutraceuticals, cosmetic preparations, body care preparations, animal feed, including pet feed.

For example, in this embodiment, the invention can be the use of a bi- or multifunctional fusion protein and/or fusion peptide, or of a system, or of a kit, each as defined here before, and in the claims, in separation and/or detection applications, in particular in the separation from and/or detection in an environment of one or more target polymers or target plastics, wherein the said environment is that of or an environment related to a production, processing, packaging, and/or bottling process and/or quality and/or safety control process of any one of food, beverages, drinking water, diets, functional food, functional beverages; preferably wherein the said environment is that of or an environment related to a production, processing, packaging, and/or bottling process and/or quality and/or safety control process of any one of food, beverages (e.g. beer, wine, fruit juice, lemonade, soft drink) and drinking water.

In another embodiment the invention relates to a method of, preferably selective, separation from an environment of one or more target polymers or target plastics, preferably fragments and/or particles thereof, comprising the steps of:
providing a bi- or multifunctional fusion protein and/or fusion peptide as defined above, and in the claims, independently, or as part of a system as defined above, and in the claims, or as part of a kit as defined above, and in the claims, comprising one or more of an adhesion promoting protein and/or adhesion promoting peptide (I), which, preferably selectively, binds to one or more of a first polymer or plastic target surface, and comprising one or more of an adhesion promoting protein and/or adhesion promoting peptide (II), which, preferably selectively, binds to one or more of a second polymer or non-polymer carrier surface;
b) providing a polymer or non-polymer carrier or carrier system as defined above, and in the claims, independently, or as part of a system as defined above, and in the claims, or as part of a kit as defined above, and in the claims;
c) providing a liquid medium comprising or potentially comprising one or more target polymers or target plastics, preferably fragments and/or particles thereof;
d) contacting the liquid medium of c) with the fusion protein and/or fusion peptide or the system of a), and allowing the binding to the fusion protein and/or fusion peptide or the system of a), of at least a part or all of the one or more target polymers or target plastics, preferably fragments and/or particles thereof, comprised in the liquid medium; and with
   the second polymer or non-polymer carrier or carrier system of b) allowing the binding to the second polymer or non-polymer carrier or carrier system b), of at least a part or all of the fusion protein and/or fusion peptide of a);
e) removing the liquid medium of c) from the fusion protein and/or fusion peptide or the system of a) and/or the second polymer or non-polymer carrier or carrier system b);
f) optionally removing at least a part or all of the one or more target polymers or target plastics, preferably fragments and/or particles thereof, bound by the fusion protein and/or fusion peptide or the system of a) to the second polymer or non-polymer carrier or carrier system b) from the said second polymer or non-polymer carrier or carrier system b);
   and optionally
g) continuously or batch-wise repeating of the steps a) to e), and/or optionally of the steps a) to f).

In still another embodiment the invention relates to a method of detection, preferably in terms of identification and/or quantification, in an environment of one or more target polymers or target particles, preferably fragments and/or particles thereof, comprising the steps of:
a) providing a bi- or multifunctional fusion protein and/or fusion peptide as defined herein, and in the claims, independently, or as part of a system as defined herein, and in the claims, or as part of a kit as defined herein, and in the claims, comprising one or more of an adhesion promoting protein and/or adhesion promoting peptide (I) which, preferably selectively, binds to one or more of a first polymer or plastic target surface, and comprising one or more of an adhesion promoting protein and/or adhesion promoting peptide (II), which, preferably selectively, binds to one or more of a second polymer or non-polymer carrier surface, and further comprising one or more of a function for generating one or more of a signal;
b) providing a second polymer or non-polymer carrier or carrier system as defined herein, and in the claims, independently, or as part of a system as defined herein, and in the claims, or as part of a kit as defined herein, and in the claims;
c) providing a liquid medium comprising or potentially comprising one or more target polymers or target plastics, preferably fragments and/or particles thereof;
d) contacting the liquid medium of c) with
   the fusion protein and/or fusion peptide or the system of a), and allowing the binding to the fusion protein and/or fusion peptide or the system of a), of at least a part or all of the one or more target polymers or target plastics, preferably fragments and/or particles thereof, comprised in the liquid medium; and with
   the second polymer or non-polymer carrier or carrier system of b) allowing the binding to the second polymer or non-polymer carrier or carrier system b), of at least a part or all of the fusion protein and/or fusion peptide of a);
e) removing the liquid medium of c) from the fusion protein and/or fusion peptide or the system of a) and/or from the second polymer or non-polymer carrier or carrier system b); and
f) optionally removing at least a part or all of the one or more target polymers or target plastics, preferably fragments and/or particles thereof, bound by the fusion protein and/or fusion peptide or the system of a) to the second polymer or non-polymer carrier or carrier system b) from the said second polymer or non-polymer carrier or carrier system b);
g) detecting the one or more target polymers or target plastics, preferably fragments and/or particles thereof, bound to the fusion protein and/or fusion peptide or the system of a) by means of the function for generating one or more of a signal comprised in a).

In an embodiment the invention also relates to a method of preparing a bi- or multifunctional fusion protein and/or fusion peptide as defined herein, and in the claims, preferably for use in the, preferably selective separation from and/or detection, preferably in terms of identification and/or quantification, in an environment of one or more target polymers or target plastics, preferably fragments and/or particles thereof, comprising the steps of:
a) providing the following of
   (i) one or more of a first adhesion promoting protein and/or adhesion promoting peptide (I), as defined herein, and in the claims, which, preferably selectively, binds to one or more of a first polymer or plastic target surface; and
   (ii) one or more of a second adhesion promoting protein and/or adhesion promoting peptide (II), as defined herein, and in the claims, which, preferably selectively, binds to one or more of a second polymer or non-polymer carrier surface;
   and of
   (iii) a spacer unit between the first and the second adhesion promoting protein and/or adhesion promoting peptide, whereby the first and
      the second adhesion promoting protein and/or adhesion promoting peptide are bonded together, preferably covalently, by the said spacer unit;
      wherein the spacer units are a flexible or rigid and/or stiff, or have any degree of mobility between being flexible or rigid and/or stiff, and/or are a cleavable linker;
      and preferably wherein the spacer unit comprises or is composed of a unstructured and therefore flexible peptide sequence, or comprises or is composed of a stiff secondary structure element of a peptide, preferentially a stiff helical peptide structure, and/or wherein the spacer unit comprises or is composed of a large spacer unit selected from a polypeptide and/or protein as defined above, preferably wherein the spacer unit a large spacer unit is selected from a separator protein;
   and/or optionally of
   (iv) one or more of a function for generating one or more of a signal;
b) providing a ligation method, preferably a PLICing (Phosphorothioate-based ligase-independent gene cloning) method;
c) fusing of the one or more of a first adhesion promoting protein and/or adhesion promoting peptide provided in (i) and the one or more of a first adhesion promoting protein and/or adhesion promoting peptide provided in (ii), by the ligation method b);
and of
d) carrying out fusion step c) such that a spacer unit provided in (iii) is fused in between the first, provided in (i), and the second, provided in (ii), adhesion promoting protein and/or adhesion promoting peptide, whereby the first provided in (i) and the second provided in (ii) adhesion promoting protein and/or adhesion promoting peptide are bonded together, preferably covalently, by the said spacer unit (iii);
and/or optionally of
e) fusing one or more of a function (iv) for generating one or more of a signal, before or after carrying out any of the fusion steps c) and/or d);
and
f) collecting, and optionally purifying, the bi- or multifunctional fusion protein and/or fusion peptide.

In the following, for purpose of further illustration, it shall be described how the present invention can, for example, solve problems of the state of the art.

As an example, the invention describes two novel protein-based systems for the, preferably selective, separation and/or detection of microplastics (MP, particle size less than 5 mm). In this case, for example, the adhesion promoter protein has a directing effect due to the specific binding properties of the selective or optimized for each application. The spacer serves as a spatial separation between adhesion promoter protein and catalytic component and the length of the spacer can be optimized according to the desired application.

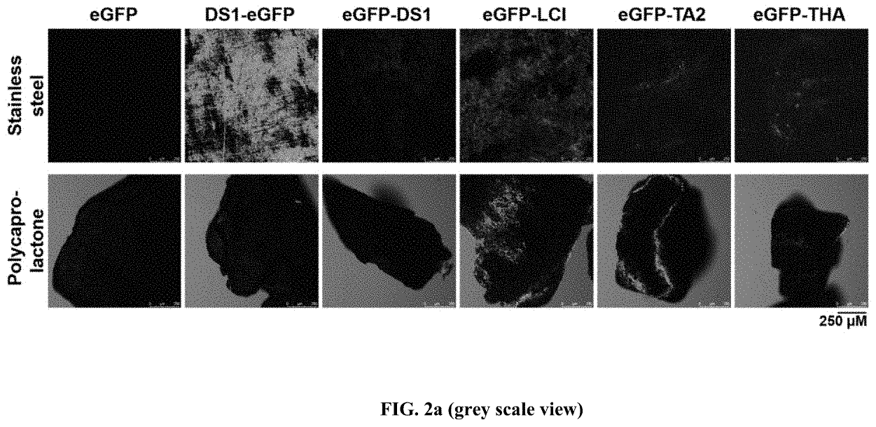

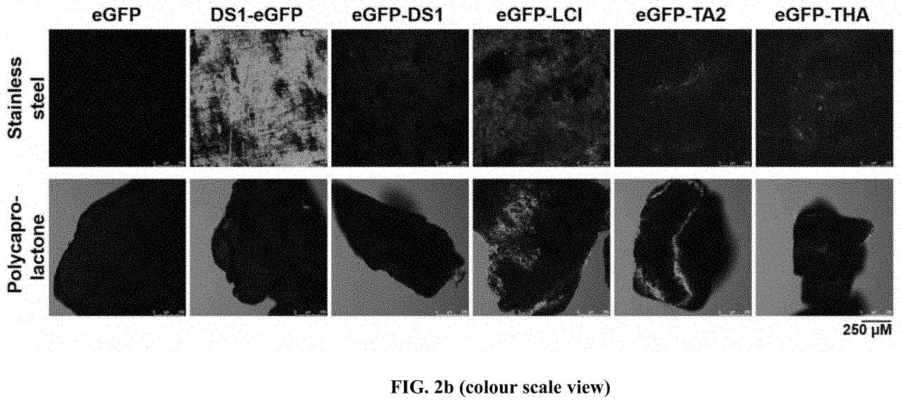

The protein-based system for the separation or detection of MP aims at all common MP polymers, so in addition to MP on PE, PP and PU-based on MP based on, for example, polystyrene (PS), polyvinyl chloride (PVC), Polycarbonate (PC), polyamide (PA), polyoxymethylene (POM), polymethyl methacrylate (PMMA), polyethylene terephthalate (PET), polybutylene terephthalate (PBT), polytetrafluoroethylene (PTFE), polyhydroxyalkanoates (PHA), polyhydroxybutyrate (PHB), polyimide (PI), Polylactide (PLA), polyvinylidene fluoride (PVDF) and polyether ketones (PEK etc.). It consists of bi- or multifunctional fusion proteins (see FIG. 2) which on the one hand have at least one function for binding the microplastic surface (adhesion promoter protein) and on the other hand at least one function for attachment to a carrier or at least one function for signal generation. The carrier system is characterized by easy separability and/or is part of a filter system. The signal generation function (s) may, for example, consist of a protein sequence which is detectable by fluorescence or which binds a dye. FIG. 2 elucidates by way of a schematic representation of the protein-based system for removing MP by selective separation of MP consisting of bifunctional fusion proteins in the example set forth, which on the one hand have a function for binding the microplastic surface and on the other hand a function for immobilization on a support. When targeting MP detection, a signal generation function is used instead of a carrier immobilization function.

The function(s) for binding the microplastic surface is/are comparable to the adhesion promoter protein of the system explained above and is aimed at the preferred binding to MP. Targeting the selective separation of MP uses the function(s) to attach to a support. The function(s) for immobilization to a carrier has/have amino acids which either bind directly to the carrier or which are suitable, for example, for chemical conjugation to the carrier or for other immobilization methods. The fact that the carrier can be easily separated or is part of a filter system (coating of, for example, ceramic or cloth filters) also separates the microplastic particles bound to the bi- or multifunctional proteins.

If the detection of MP is targeted, a function or functions will be used for signal generation, which may for example consist of a protein sequence/which z. B. is detectable via fluorescence, or which binds a dye. It is also possible to fuse an enzymatic component (with or without linker) to the function (s) for binding the microplastic surface, so that a simple for example, color-based enzyme activity assay an easily detectable signal can be generated. By generating an easy-to-capture signal (such as color), it is possible to detect the presence of MP comparatively easily and quickly. Alternative systems for quantifying MP levels via the protein-based system of the invention shown herein include physical methods, e.g. QCM-D (quartz crystal microbalance with dissipation monitoring).

Thus, on the one hand, it is possible to determine the total MP content and, on the other hand, by selecting the function (s) for binding the microplastic surface, it is also possible to detect specific MP varieties (such as PP and PE, which can bind toxic substances).

In the following, for purpose of further illustration, it shall be described what added value the present invention provides over the state of the art.

A significant advantage of the invention is their directional or preferred binding of MP via the adhesion promoter protein or the function(s) for binding to the microplastic surface, so that the MP is selectively separated or can be detected. The detection is much easier and less expensive to achieve by the function(s) for signal generation than in most other available methods for the detection of MP. In addition, the sample preparation is less expensive due to the selectivity of the system according to the invention. By fine-tuning the composition of the function(s) for binding the microplastic surface, on the one hand, it is possible to determine the total MP content and, on the other hand, it is also possible to detect specific types of MP (such as PP and PE, which can bind toxic substances).

Furthermore, e.g. in terms of safety and environmental considerations, the invention provides a prominent advantage, over the protein-based systems described in the prior art which merely focus on the degradation of MP. For example, besides detecting specific types of polymers and/or plastics, in particular of MP, e.g. such as PP and PE, which can bind toxic substances, by means of the invention also such polymers and/or plastics, in particular of MP, are addressed that are normally not or only very difficult to break down. Here, the invention provides the prominent advantage in that toxic substances are highly bound and not arbitrarily released again from the adhesion promoter protein or the function(s) that is binding to the microplastic surface. Thereby, e.g. by separating the polymers and/or plastics, in particular of MP, from an environment the toxic substances remain safely bound, and the contaminated polymers and/or plastics, in particular of MP, can be easily handled and processed to safe, intermediate storage disposal, and eventually to occupationally safe and environmentally friendly disposal and/or decomposition. Therefore, the particularly difficult-to-degrade polymers and/or plastics, in particular MP, e.g. the plastics PP and PE, that contain plasticizers, which often show hormonal effects, or may contain additives which are carcinogenic, toxic or show endocrine activity, or which due to their hydrophobicity (e.g. PP and PE) bind ubiquitously occurring pollutants and/or highly dangerous contaminants such as PCBs (polychlorinated biphenyls), PAHs (polyaromatic hydrocarbons) or pesticides such as DDT (dichlorodiphenyl trichloroethane), can be advantageously separated off an environment, and then advantageously processed to safe and environmentally friendly disposal and/or decomposition.

The invention embodiments are scalable in principle and can be used in various application options.

A principal advantage of the invention embodiments is their high protein content, so that renewable resources can be used for their production and the anchor peptides I and/or anchor peptides II, and as for example contained in related systems and/or kits of the invention, are biodegradable to a high degree.

Also the anchor peptides I and/or anchor peptides II, the related systems and/or kits of the invention are suitable in a variety of applications, as for example wastewater treatment, drinking water treatment, water purification and filtration in general. Fish farming (ingested MP), food control (e.g., honey, beer, mussels), detection and quantification of MP in general, as a few exemplifications amongst others, e.g. those already mentioned above.

Therefore, the present invention can provide benefits, for example, to operators of sewage and/or drinking water treatment plants, water filter manufacturers, operators of managed waters such as fish rearing facilities, food inspectors and/or safety officers, and finally the consumer or consumer protection associations, as a few exemplifications amongst others, e.g. those already mentioned above.

The invention shall be further described and exemplified by the following examples.

### EXAMPLES

### Example 1

### Immobilization of Polycaprolactone (PCL) Particles on Stainless Steel by Adhesion-Promoting Bifunctional Peptides

The immobilization of polycaprolactone (PCL) particles on stainless steel is shown.

The bonding of both components by peptides represents a simple and environmentally friendly method. Anchor peptides are naturally occurring short peptides which, apart from their natural property of being able to integrate into membranes of microorganisms, can bind to various polymer surfaces. The peptide Cecropin A (Steiner, Hultmark et al., 1981) is capable of binding to the triblock polymer P1B1000-PEG6000-P1B1000 ((Noor, Dworeck et al., 2012, Klermund, Poschenrieder et al., 2016)). Tachystatin A2 (TA2, (Osaki, Omotezako et al., 1999)) and LCI (Gong et al., 2011) show an increased tendency to bind to polystyrene and polypropylene (Rübsam, Weber et al., 2017), (Rübsam, Stomps et al. 2017)). The use of these peptides for surface functionalization allows the generation of bifunctional peptides fused to two single anchor peptides. Bifunctional proteins allow a highly specific selection of binding properties for the union of two components by means of adhesion. Furthermore, the bonding takes place at room temperature without the use of solvents or other environmentally harmful components. In addition, they allow the use of different materials, of organic or inorganic origin. The use of molecular biological methods for protein optimization additionally allows adaptation to application conditions such as UV or ethanol sterilization.

FIG. 1: Schematic immobilization of PCL particles (could be microplastic) on stainless steel. Bifunctional fusion proteins immobilize PCL particles on the smooth steel surface. The N-terminal peptide DS1 binds to the stainless steel, whereas the C-terminal LCI binds the PCL particles. The bifunctional fusion protein serves as an adhesion promoter.

### Example 2

### Selection of Anchors for Bifunctional Proteins

The bifunctional proteins were prepared with a separator protein (domain Z). This separator allows a spatial separation and thus functionality of the two peptide components. The binding behavior of the individual peptides was analyzed by the reporter protein eGFP. Based on previous work, the anchors were fused to the C-terminus of the eGFP (Rubsam, Weber et al., 2017), (Rübsam, Stomps et al., 2017), (Meurer, Kemper et al., 2017). It has been shown that LCI (Gong, Wang et al., 2011), TA2 (Osaki, Omotezako et al., 1999) and THA (Fehlbaum, Bulet et al., 1996) show strong binding affinity to tested polymers (polypropylene/polystyrene) as well as to the leaf surfaces of plants. DS1 (Brand, Leite et al., 2002) was tested in both orientations, both N-terminal anchor and C-terminal anchor in the eGFP fusion protein.

FIG. 2: Binding of eGFP anchor peptides to stainless steel and PCL. The binding of the eGFP anchor peptides to stainless steel and PCL was tested by incubation (10 min, room temperature) on the surface. In the successive washing steps (1 mL each of ddH20) and a washing step with the detergent LAS (0.5 M, 5 min), non-specifically bound proteins were removed from the surface and the remaining bound peptides were detected via the fluorescence marker eGFP (Leica TCS SP8 microscope, Ex 485, Em 520, amplifier voltage at detector 800, Leica Microsystems GmbH (Wetzlar, Germany).

After washing the samples with LAS (0.5 mM in 50 mM Tris/HCl pH 8.0), the N-terminal DS1-eGFP showed a significantly stronger binding to the stainless steel surface in the fluorescence analysis than all the other fusion proteins tested. As expected, the negative control eGFP (without anchor) could no longer be detected on the stainless steel surface. Fluorescence analysis on the PCL surface revealed that the C-terminal anchors strongly bound eGFP-LCI, eGFP-TA2, and eGFP-THA to the surface. In contrast, the N-terminal DS1-eGFP showed no binding to PCL. The negative control eGFP (without anchor) could also no longer be detected on the PCL surface.

### Example 3

### Preparation of Bifunctional Fusion Proteins

Based on the binding analyzes, three bifunctional fusion proteins were generated. For this purpose DS1 was used as the N-terminal peptide and in each case LCI, TA2 or THA as the C-terminal peptide with the aid of the PLICing method (phosphorothioate-based ligase-independent gene cloning, Blanusa, Schenk et al. Both peptides were separated by the domain Z.

For the production of the bifunctional fusion proteins, the culturing conditions were optimized and the proteins were produced in LB medium for 16 hours at 30° C. Due to their toxicity and partial lethal effect for the producing E. coli BL21-Gold (DE3) cells, the proteins were released into the culture medium and could be concentrated therefrom. Both DS1-DZ-LCI and DS1-DZ-THA could be produced in this way. DS1-DZ-TA2 could not be detected because it was not produced by the E. coli BL21-Gold (DE3) cells because of potentially high toxicity.

### Example 4

### Production of PCL Particles

The PCL particles (could be microplastic) were made by extrusion. The particle size ranged from 0.8-180 microns with 50% of all particles having a size of 13.94±0.31 microns.

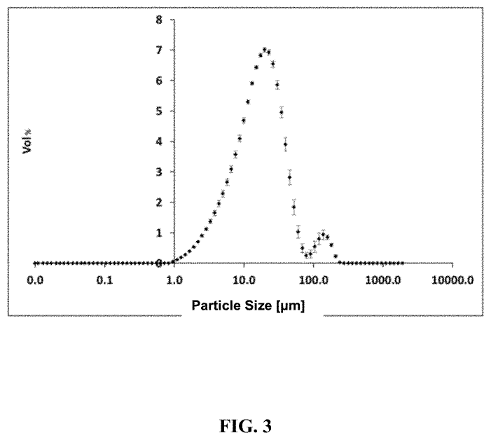

FIG. 3: Size distribution of PCL particles. The size distribution of the PCL particles was measured by Mie scattering in triplicate. Surface weighted average: 8.66±0.14 μm and volume weighted average: 20.01±0.29 μm. Measuring range 0.020-2000 μM (Micro 15 cc Twin Screw Compounder, Xplore Instruments BV, The Netherlands).

### Example 5

### Immobilization of PCL Particles on Stainless Steel

To immobilize PCL particles on stainless steel with the aid of the bifunctional peptides, the individual peptides (50 μL, three times concentrated from culture medium) and the negative control (empty vector (pET28), 50 μL, three times concentrated from culture medium) were first incubated on the stainless steel (10 min). Subsequently, the PCL particles dispersed in ethanol were added and incubated again (10 min). This was followed by three washes with 1 mL of ethanol followed by 3 washes with 1 mL each of ddH2O. The analysis of the immobilization was carried out by field emission scanning electron microscopy (FESEM).

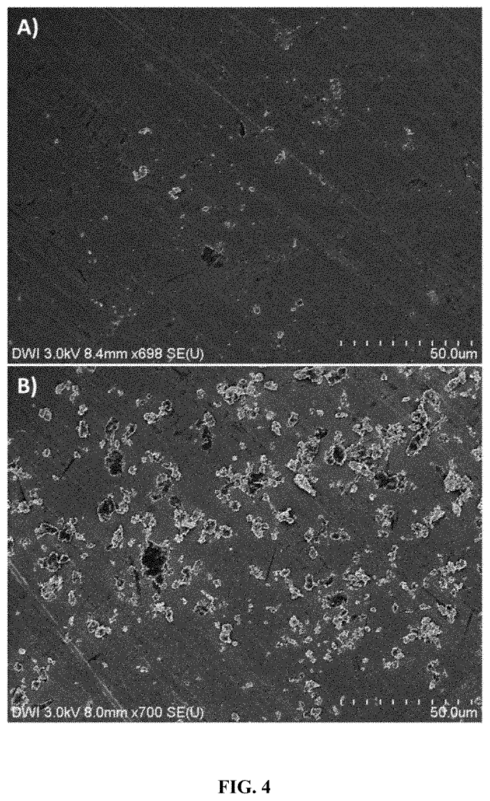

FIG. 4: FESEM analysis of DS1-DZ-LCI mediated immobilization of PCL particles on stainless steel. A) Negative control (PCL particles immobilized on stainless steel with culture supernatant of the pET28 control) at a magnification of 8.4 mm×698. B) PCL particles immobilized on stainless steel with culture supernatant of the fusion protein DS1-DZ-LCI at a magnification of 8.4 mm×700 Images were taken using a field emission scanning electron microscope (S-4800 FE-SEM, Hitachi, Schaumburg, USA). Device settings: Acceleration voltage 3 kV, working distance: 8/8.4 mm, magnification: 70/700×.

The analysis of the FESEM images showed a significantly higher number of PCL particles on the stainless steel surface when the peptide DS1-DZ-LCI was used for the immobilization. In comparison, the immobilization with the negative control resulted only sporadically to PCL particles on the surface.

### Example 6

### Identification and Selection of Anchor Peptides for Bifunctional Peptides

Anchor peptides are naturally occurring material binding peptides that offer smart and easy-handling possibilities for surface functionalization (Care, 2015; Seker, 2011). Cecropin A (Steiner, Hultmark et al. 1981) is able to bind to the triblock co-polymer PIB1000-PEG6000-PIB1000 (Noor, Dworeck et al. 2012, Klermund, Poschenrieder et al. 2016). Tachystatin A2 (TA2, (Osaki, Omotezako et al. 1999)) and LCI (Gong, Wang et al. 2011) show strong binding affinity to the biologically inert polymers polystyrene (PS) and polypropylene (PP) from aqueous solutions at ambient temperature. Binding to PP, the anchor peptide LCI showed to form a dense monolayer of 4.1 nm (in 50 mM Tris/HCl pH 8.0) (Rübsam, Stomps et al. 2017, Rubsam, Weber et al. 2018). Binding strength and specificity is tunable to application conditions by directed evolution applying the PePevo protocol (Cheng, Zhu et al. 2015, Rubsam, Weber et al. 2018).

The reporter protein eGFP (enhanced green fluorescent protein) was used to quantify anchor peptides that are bound on stainless steel or PCL surfaces. eGFP and the selected anchor peptides were separated by a stiff spacer helix (17 amino acids, as described by Arai, Ueda et al. 2001) with an incorporated TEV cleavage site (7 amino acids, as described by Kapust, Tozser et al. 2001). LCI (Liquid chromatography peak 1, 47 amino acids, as described by Gong, Wang et al. 2011), TA2 (Tachystatin A2, 44 amino acids, as described by Osaki, Omotezako et al. 1999), and THA (Thanatin, 21 amino acids, as described by Fehlbaum, Bulet et al. 1996) already proved to have a high potential as polymer-(PS and PP) and leaf surface-binders when C-terminally fused to eGFP (referred to as C-anchors, as described by Meurer, Kemper et al. 2017, Rübsam, Stomps et al. 2017, Rubsam, Weber et al. 2018). Material binding properties of DS1 (Dermaseptin 51, 29 amino acids, as described by Brand, Leite et al. 2002) have not yet been reported; therefore eGFP fusions with an N-terminal anchor (DS1-eGFP) as well as C-terminal anchor (eGFP-DS1) orientation were also investigated.

Binding of DS1-eGFP, eGFP-DS1, eGFP-LCI, eGFP-TA2, and eGFP-THA to stainless steel as well as PCL (polycaprolactone) were analyzed by confocal microscopy (see FIG. 2). DS1-eGFP showed the strongest binding to stainless steel whereas the C-terminal anchor peptide DS1 (eGFP-DS1) was removed almost completely after washing. eGFP-LCI fluorescence was distinctly decreased in comparison to DS1-eGFP. Binding analysis of PCL revealed that eGFP-LCI and eGFP-TA2 bound strongest after washing with the anionic surfactant sodium dodecylbenzenesulfonate (LAS) (0.5 mM in 50 mM Tris/HCl pH 8.0, reduction of eGFP background (Rübsam, 2018)). DS1-eGFP and eGFP-DS1 were not detectable on PCL surface after washing. Consequently, DS1 was selected as a stainless steel-binder and LCI, TA2 or THA as suitable C-terminal PCL anchor peptides.

Results of Binding of eGFP-anchor peptides to stainless steel and PCL. Binding of negative control eGFP and eGFP-anchor peptides (DS1, LCI, TA2, and THA) was investigated by incubation (10 min, ambient temperature) on stainless steel or PCL particles. Three successive washing steps (1 mL ddH20 each) followed by a washing step with LAS (0.5 mM, 0.5 mL, 5 min) were performed to prevent non-specific binding of eGFP. Binding of eGFP and anchor peptide fusion proteins was analyzed by confocal microscopy (Leica TCS SP8 microscope, Ex: 485 nm, Em: 520 nm, argon laser 20% intensity, gain 1000, Leica Microsystems GmbH (Wetzlar, Germany).

### Production of Fusion Anchor Peptides:

Fusion anchor peptides consisted of two functional anchor peptides, separated by the stiff staphylococcal protein A domain Z (DZ, 58 aa). DZ is a-helical protein forming three antiparallel helices and therefore the N- and C-terminus of the protein are located on opposite sides of the domain (Tashiro, Tejero et al. 1997). Generated fusion anchor peptides were composed of the N-terminal anchor peptide DS1, the separator DZ, and the C-terminal anchor peptide LCI, TA2, or THA.

Anchor peptides used in this study belong to the class of antimicrobial peptides. Due to their potential toxicity to the host organism and their small size (<15 kDa), the production can be challenging (Chen, Li et al. 2017, Khosa, Scholz et al. 2018). Additionally, antimicrobial peptides tend to attach to bacterial membranes (Brogden 2005) and are therefore often found in crude extracts after cell lysis. The spacer protein DZ was used to separate and increase solubility of fusion anchor peptide (Samuelsson, Moks et al. 1994). Additions ally the expression of DS1-DZ-LCI, DS1-DZ-TA2, and DS1-DZ-THA in E. coli BL21-Gold (DE3) was optimized by varying expression temperature (20, 30, 37° C.) and time (16 h and 48 h) (finally used: 30° C., 16 h, 200 rpm, 70%, humidity, 50 mL LB medium). An expression temperature of 30° C. was chosen as suitable balance between soluble fusion anchor peptide expression and cell viability. DS1-DZ-TA2 could not be expressed in SDS-detectable amounts at any temperature. The fusion anchor peptide DS1-DZ-LCI and DS1-DZ-THA were enriched from culture broth and dialyzed against water for binding studies without further purification steps (EMD Millipore Amicon™ Ultra-0.5 Centrifugal Filter Units, MWCO 10 kDa, Thermo Fisher Scientific (Darmstadt, Germany)).

### Binding of Fusion Anchor Peptides to PCL and Stainless Steel

The ability of fusion anchor peptides (DS1-DZ-LCI, DS1-DZ-TA2, and DS1-DZ-THA) as well as the negative control (pET28-EV) to function as binding promotor was analyzed by incubation of the fusion anchor peptides with cl-PCL and stainless steel.

Fusion anchor peptides mediated immobilization of PCL and on stainless steel was investigated by confocal microscopy and FE-SEM analysis.

Results of fusion anchor peptide mediated binding of PCL particles and stainless steel. A) Morphology and size distribution of PCL in ethanol determined by confocal analysis (Leica TCS SP8 microscope; 63-fold magnification, zoom 2, PMT Trans detector, gain 300, Leica Microsystems GmbH (Wetzlar, Germany)) and Mie-scattering (Mastersizer 2000; Size range 0.020-2000 μm, Malvern Panalytical GmbH (Kassel, Germany)). B-C) Field emission scanning electron microscopy analysis of assembly. B) PCL bound on stainless steel with negative control (pET28-EV). C) PCL bound on stainless steel with DS1-DZ-LCI. (S-4800 FE-SEM; accelerating voltage: 3 kV, working distance: 8.0/8.4 mm, magnification: 700×, Hitachi, (Schaumburg, USA)).

The highest amount of PCL bound on stainless steel was achieved with DS1-DZ-LCI (confocal analysis). The negative control (confocal analysis) did not show any PCL binding on stainless steel wires. DS1-DZ-THA showed a lower binding strength in comparison to DS1-DZ-LCI (confocal analysis).

A detailed analysis of binding was performed by FE-SEM using DS1-DZ-LCI and the negative control pET28-EV. After stainless steel coating, the surface was sputtered with a 4 nm layer of Au—Pd to avoid electrostatic effects. FE-SEM analysis confirmed that only the fusion anchor peptide DS1-DZ-LCI is able to bind efficiently PCL on stainless steel. Micro-containers detected on the surface ranged in size from <5-25 μm. Particle size of PCL was determined by Mie-scattering to be 13.94±0.31 μm (surface weighted mean (d 0.5), which is in good correlation to the observed micro-containers on the stainless steel surface. Immobilization using the control pET28-EV showed as expected that only few PCL micro-containers (<10 μm) were bound on the stainless steel surface.

### Example 7

### Detection of Microparticles (Microplastic)

### Microparticle (Microplastic) Detection:

For the detection of polymers (microplastic), anchor peptides were genetically fused to a reporter protein (green fluorescent protein; eGFP) and an enzyme (phytase; ymPhy). The polymer is detected by the green fluorescence of eGFP or by the conversion of a non-fluorescent substrate into a fluorescent product catalyzed by the fused phytase.

### Used Anchor Peptides, Reporter Proteins and Enzymes:

The enhanced green fluorescent protein (EGFP) (Rübsam, K., Stomps, B., Böker, A., Jakob, F., Schwaneberg, U. (2017). Anchor peptides: A green and versatile method for polypropylene functionalization. Polymer, 116, 124-132), and the Yersinia mollaretii phytase (ymPhy) was fused genetically to three selected anchor peptides:
(1) CecA: Cecropin A from organism Hyalophora cecropia; see Steiner, H., D. Hultmark, A. Engstrom, H. Bennich and H. G. Boman (1981). “Sequence and specificity of two antibacterial proteins involved in insect immunity.” Nature 25 292(5820): 246-248);
(2) LCI: Liquid chromatography peak from organism; see Gong, W., J. Wang, Z. Chen, B. Xia and G. Lu (2011). “Solution structure of LCI, a novel antimicrobial peptide from Bacillus subtilis.” Biochemistry 50 (18): 3621-3627; and
(3) TA2: Tachystatin A2 from organism Limulus Polyphemus; see Osaki, T., M. Omotezako, R. Nagayama, M. Hirata, S. Iwanaga, J. Kasahara, J. Hattori, I. Ito, H. Sugiyama and S. Kawabata (1999). “Horseshoe crab hemocyte-derived antimicrobial polypeptides, tachystatins, with sequence similarity to spider neurotoxins.” J Biol Chem 274(37): 26172-26178.

EGFP and ymPhy were separated by a stiff spacer helix from composed of 17 amino acids (AEAAAKEAAAKEAAAKA) (Arai, R., Ueda, H., Kitavama, A., Kamiya, N., & Naqamune, T. (2001). Design of the linkers which effectively separate domains of a bifunctional fusion protein. Protein engineering, 14(8), 529-532) from the anchor peptides. Fusion constructs were recombinantly produced in E. coli.

### Recombinant Production of Fusion Proteins and Fusion Enzymes:

For preculture preparation, 4 mL LB-media (10 g/L tryptone, 10 g/L NaCl, 5 g/L yeast extract, 50 μg/mL ampicillin) were inoculated with the corresponding cryoculture and incubated (16 h, 37° C., 210 rpm; Multitron Pro, Infors AG, Bottmingen, Switzerland) in glass tubes. Main cultures containing 100 mL TB-media (24 g/L yeast extract, 12 g/L peptone, 4 mL/L glycerol, 12.54 g/L K₂HPO₄, 2.31 g/L KH₂PO₄, appropriate antibiotic) were inoculated with 1 mL preculture and incubated (37° C., 2 h, 210 rpm; Multitron Pro, Infors AG). Protein overexpression was induced by adding isopropyl β-D-1-thiogalactopyranoside (IPTG, 0.1 mM final concentration) and reduction in cultivation temperature to 30° C. (incubation: 4 h, 210 rpm; Multitron Pro, Infors AG). The cells were harvested by centrifugation (3,200 g, 20 min, 4° C.; Eppendorf centrifuge 5810 R, Eppendorf AG, Hamburg, Germany) and the pellets were stored at −20° C.

Polymer Detection Using eGFP Anchor Peptide Fusion Proteins:

Frozen cell pellets were suspended in Tris-HCl buffer (50 mM, pH 8.0; 6 mL buffer on 1 g cell pellet). Cell lysis was performed with ultrasonication (3 min, pulse 15/15, 60% amplitude; ultrasonic processor VCX 130, Sonics & Materials Inc., Newton, USA) and centrifuged (21,130 g, 15 min, 4° C.; Eppendorf centrifuge 5424 R) to separate the soluble protein from insoluble proteins and cell fragments. The investigated polymers (PP, PS, and PET as plane surface and microparticles) were coated with eGFP anchor peptide fusion proteins (50 μL supernatant 15 min, RT). As control, supernatant containing EGFP-17H-TEV without anchor peptide was investigated. According to previous work, saturated conditions were utilized (Rübsam et al., 2017). Supernatant was removed, the materials rinsed with 10 mL of Tris-HCl buffer (50 mM, pH 8.0), transferred into squared petri dishes filled with 2 mL of Tris-HCl buffer (50 mM, pH 8.0). Samples were incubated (2 min), buffer removed and new buffer added (three times). The samples were removed, rinsed with 10 mL of ddH₂0 and dried with nitrogen flow. Binding of EGFP-anchor peptide fusion proteins was determined by confocal fluorescence microscopy (TCS SP8, Leica Microsystems CMS GmbH, Mannheim, Germany). Samples were excited with 488 nm, 10% laser intensity. Detection was performed with a PMT2 detector (emission 500-565 nm, varied gain for each material).

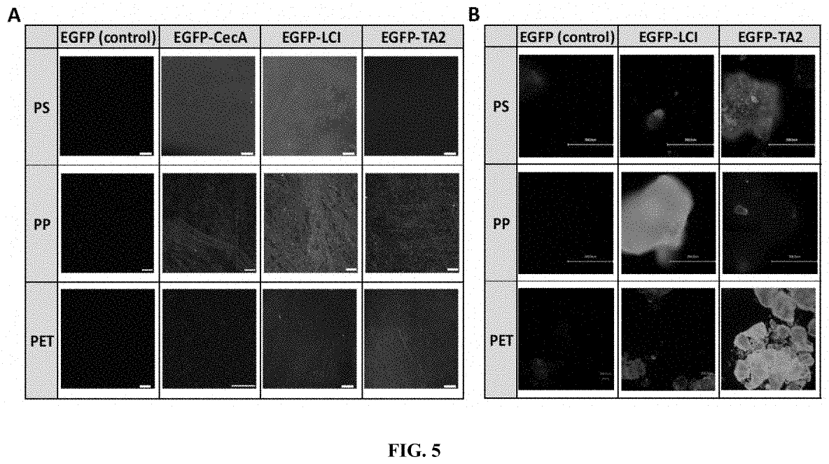

For the results obtained see FIG. 5. FIG. 5 refers to the binding of EGFP-anchor peps tide fusion proteins to the analysed polymer materials that was determined by confocal fluorescence microscopy. A) of FIG. 5 shows results for PP, PS, and PET as plane surface and B) of FIG. 5 shows results for PP, PS, and PET microparticles. As negative control EGFP-17H-TEV (without anchor peptide) was used, to determine unspecific binding. Briefly, the negative control displayed no fluorescence on any material under the applied washing conditions. For every tested polymer a suitable anchor peptide for polymer detection was identified.

### Polymer Detection Using Phytase-Anchor Peptide Fusion Proteins:

The activity of ymPhy and ymPhy-anchor peptide fusion proteins was determined with the 4-methylumbelliferyl-3-D-phosphate (4-MUP) assay (see Shivange, A. V., Serwe, A., Dennig, A., Roccatano, D., Haefner, S., & Schwaneberg, U. (2012). Directed evolution of a highly active Yersinia mollaretii phytase. Applied Microbiology and Biotechnology, 95(2), 405-418. doi:10.1007/s00253-011-3756-7). Phytase hydrolyzes the non-fluorescent 4-MUP to the fluorescent product 4-MU. The substrate 4-MUP is prepared as 10 mM stock solution in 250 mM sodium acetate buffer (250 mM sodium acetate, pH 5.5, 1 mM CaCl₂), 0.01%, Tween-20). For activity assays, the stock solution is diluted to 1 mM 4-MUP with sodium acetate buffer (250 mM sodium acetate, pH 5.5, 1 mM CaCl₂), 0.01% Tween-20).

For the activity detection of bound ymPhy-anchor peptide fusion proteins on PS and PP, 50 μL of 1 mM 4-MUP solution was added onto the dry wells. The activity was monitored using Tecan Infinite® M1000 PRO microtiter plate reader (gain 140; interval 1 min, time 20 min, temperature RT, λₑₓ 360 nm, λₑₘ 465 nm). To determine the activity on the substrates PET, the reaction was started by addition of 300 μL 4-MUP to each sample (1 mM, 250 mM sodium acetate, pH 5.5, 1 mM CaCl₂), 0.01% Tween-20). Reaction solution (25 μL) was transferred to black PS MTPs and diluted with sodium acetate buffer (25 μL; 250 mM sodium acetate, pH 5.5, 1 mM CaCl₂), 0.01% Tween-20). Activity was monitored utilizing Tecan Infinite® M1000 PRO (interval 30 min, time 180 min, λₑₓ 330 nm, λₑₘ 450 nm, gain 140, RT).

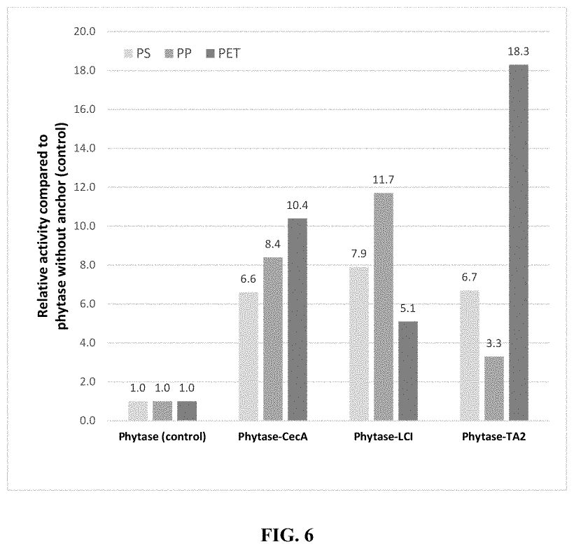

For the results obtained see FIG. 6. FIG. 6 refers to the phytase reporter enzyme that was immobilized by the anchor peptides CecA, LCI, and TA2 on the target polymers (PS, PP, and PET) and the activity that was determined using the fluorescent 4-MUP assay. Compared to the phytase wild type all phytase fusion enzymes showed a significantly improved fluorescent signal allowing the detection of microplastic particles.

### LIST OF REFERENCES

Amet, N., H. F. Lee and W. C. Shen (2009). “Insertion of the designed helical linker led to increased expression of tf-based fusion proteins.” Pharm Res 26(3): 523-528.
Arai, R., H. Ueda, A. Kitayama, N. Kamiya and T. Nagamune (2001). “Design of the linkers which effectively separate domains of a bifunctional fusion protein.” Protein Eng 14(8): 529-532.
Argos, P. (1990). “An investigation of oligopeptides linking domains in protein tertiary structures and possible candidates for general gene fusion.” J Mol Biol 211(4): 943-958.
Blanusa, M., A. Schenk, H. Sadeghi, J. Marienhagen and U. Schwaneberg (2010). “Phosphorothioate-based ligase-independent gene cloning (PLICing): An enzyme-free and sequence-independent cloning method.” Anal Biochem 406(2): 141-146.
Bose, S., M. Roy and A. Bandyopadhyay (2012). “Recent advances in bone tissue engineering scaffolds.” Trends Biotechnol 30(10): 546-554.
Brand, G. D., J. R. Leite, L. P. Silva, S. Albuquerque, M. V. Prates, R. B. Azevedo, V. Carregaro, J. S. Silva, V. C. Sa, R. A. Brandao and C. Bloch, Jr. (2002). “Dermaseptins from Phyllomedusa oreades and Phyllomedusa distincta. Anti-Trypanosoma cruzi activity without cytotoxicity to mammalian cells.” J Biol Chem 277(51): 49332-49340.
Chapman, S., C. Faulkner, E. Kaiserli, C. Garcia-Mata, E. I. Savenkov, A. G. Roberts, K. J. Oparka and J. M. Christie (2008). “The photoreversible fluorescent protein iLOV outperforms GFP as a reporter of plant virus infection.” Proceedings of the National Academy of Sciences 105(50): 20038.
Chen, X., J. Zaro and W.-C. Shen (2013). “Fusion Protein Linkers: Property, Design and Functionality.” Advanced drug delivery reviews 65(10): 1357-1369.
Cormack, B. P., R. H. Valdivia and S. Falkow (1996). “FACS-optimized mutants of the green fluorescent protein (GFP).” Gene 173(1 Spec No): 33-38.
Duvick, J. P., T. Rood, A. G. Rao and D. R. Marshak (1992). “Purification and characterization of a novel antimicrobial peptide from maize (Zea mays L.) kernels.” J Biol Chem 267(26): 18814-18820.
Ehret-Sabatier, L., D. Loew, M. Goyffon, P. Fehlbaum, J. A. Hoffmann, A. van Dorsselaer and P. Bulet (1996). “Characterization of novel cysteine-rich antimicrobial peptides from scorpion blood.” J Biol Chem 271(47): 29537-29544.
Fehlbaum, P., P. Bulet, S. Chernysh, J. P. Briand, J. P. Roussel, L. Letellier, C. Hetru, and J. A. Hoffmann (1996). “Structure-activity analysis of thanatin, a 21-inducible insect defense peptide with sequence homology to frog skin antimicrobial peptides.” Proc Natl Acad Sci USA 93 (3): 1221-1225.
Glaser, R., J. Harder, H. Lange, J. Bartels, E. Christophers and J. M. Schroder (2005). “Antimicrobial psoriasin (S100A7) protects human skin from Escherichia coli infection.” Nat Immunol 6(1): 57-64.
Gong, W., J. Wang, Z. Chen, B. Xia and G. Lu (2011). “Solution structure of LCI, a novel antimicrobial peptide from Bacillus subtilis.” Biochemistry 50 (18): 3621-3627.
Griesbeck, O., G. S. Baird, R. E. Campbell, D. A. Zacharias and R. Y. Tsien (2001). “Reducing the environmental sensitivity of yellow fluorescent protein. Mechanism and applications.” J Biol Chem 276(31): 29188-29194.
Gueguen, Y., A. Herpin, A. Aumelas, J. Gamier, J. Fievet, J. M. Escoubas, P. Bulet, M. Gonzalez, C. Lelong, P. Favrel and E. Bachere (2006). “Characterization of a defensin from the oyster Crassostrea gigas. Recombinant production, folding, solution structure, antimicrobial activities, and gene expression.” J Biol Chem 281(1): 313-323.
Hakan, S., Andreau, D., Merrifield, R. B. (1988). Binding and action of Cecropin and Cecropin analogues: antibacterial peptides from insects. Biochim. Biophys. Acta 939, 260-266.
Iravani, A., A. D. Tice, J. McCarty, D. H. Sikes, T. Nolen, H. A. Gallis, E. P. Whalen, R. L. Tosiello, A. Heyd, S. F. Kowalsky and et al. (1995). “Short-course ciprofloxacin treatment of acute uncomplicated urinary tract infection in women. The minimum effective dose. The Urinary Tract Infection Study Group [corrected].” Arch Intern Med 155(5): 485-494.John, T., A. Rajpurkar, G. Smith, M. Fairfax and J. Triest (2007). “Antibiotic pretreatment of hydrogel ureteral stent.” J Endourol 21(10): 1211-1216.
Kapust, R. B., J. Tozser, J. D. Fox, D. E. Anderson, S. Cherry, T. D. Copeland and D. S. Waugh (2001). “Tobacco etch virus protease: mechanism of autolysis and rational design of stable mutants with wild-type catalytic proficiency.” Protein Eng 14(12): 993-1000.
Klement, M., C. Liu, B. L. W. Loo, A. B.-H. Choo, D. S.-W. Ow and D.-Y. Lee (2015). “Effect of linker flexibility and length on the functionality of a cytotoxic engineered antibody fragment.” Journal of Biotechnology 199: 90-97.
Klermund, L., S. T. Poschenrieder and K. Castiglione (2016). “Simple surface functionalization of polymersomes using non-antibacterial peptide anchors.” J Nanobiotechnol 14(48): 1-12.
Niccoli, G., G. A. Sgueglia, R. A. Montone, M. Roberto, A. P. Banning and F. Crea (2014). “Evolving management of patients treated by drug-eluting stent: prevention of late events.” Cardiovasc Revasc Med 15(2): 100-108.
Nissen-Meyer, J., A. G. Larsen, K. Sletten, M. Daeschel and I. F. Nes (1993). “Purification and characterization of plantaricin A, a Lactobacillus plantarum bacteriocin whose activity depends on the action of two peptides.” J Gen Microbiol 139(9): 1973-1978.
Meurer, R. A., S. Kemper, S. Knopp, T. Eichert, F. Jakob, H. E. Goldbach, U. Schwaneberg and A. Pich (2017). “Biofunctional Microgel-Based Fertilizers for Controlled Foliar Delivery of Nutrients to Plants.” Angew Chem Int Ed Engl 56(26): 7380-7386.
Noor, M., T. Dworeck, A. Schenk, P. Shinde, M. Fioroni and U. Schwaneberg (2012). “Polymersome surface decoration by an EGFP fusion protein employing Cecropin A as peptide “anchor”.” J Biotechnol 157(1): 31-37.
Osaki, T., M. Omotezako, R. Nagayama, M. Hirata, S. Iwanaga, J. Kasahara, J. Hattori, I. Ito, H. Sugiyama and S. Kawabata (1999). “Horseshoe crab hemocyte-derived antimicrobial polypeptides, tachystatins, with sequence similarity to spider neurotoxins.” J Biol Chem 274(37): 26172-26178.
Quent, V., A. V. Taubenberger, J. C. Reichert, L. C. Martine, J. A. Clements, D. W. Hutmacher and D. Loessner (2017). “A humanised tissue-engineered bone model allows species-specific breast cancer-related bone metastasis in vivo.” J Tissue Eng Regen Med.
Rizzo, M. A., G. H. Springer, B. Granada and D. W. Piston (2004). “An improved cyan fluorescent protein variant useful for FRET.” Nat Biotechnol 22(4): 445-449.
Rodriguez, E. A., G. N. Tran, L. A. Gross, J. L. Crisp, X. Shu, J. Y. Lin and R. Y. Tsien (2016). “A far-red fluorescent protein evolved from a cyanobacterial phycobiliprotein.” Nat Methods 13(9): 763-769.
Rübsam, K., B. Stomps, A. Böker, F. Jakob and U. Schwaneberg (2017). “Anchor peptides: A green and versatile method for polypropylene functionalization.” Polymer 116: 124-132.
Rübsam, K., L. Weber, F. Jakob and U. Schwaneberg (2017). “Directed evolution of polypropylene and polystyrene binding peptides.” Biotechnol Bioeng.
Schittek, B., R. Hipfel, B. Sauer, J. Bauer, H. Kalbacher, S. Stevanovic, M. Schirle, K. Schroeder, N. Blin, F. Meier, G. Rassner and C. Garbe (2001). “Dermcidin: a novel human antibiotic peptide secreted by sweat glands.” Nat Immunol 2(12): 1133-1137.
Schulte, S. (2009). “Half-life extension through albumin fusion technologies.” Thromb Res 124 Suppl 2: S6-8.
Shaner, N. C., P. A. Steinbach and R. Y. Tsien (2005). “A guide to choosing fluorescent proteins.” Nat Methods 2(12): 905-909.
Shao, F., Z. Hu, Y. M. Xiong, Q. Z. Huang, WangCg, R. H. Zhu and D. C. Wang (1999). “A new antifungal peptide from the seeds of Phytolacca americana: characterization, amino acid sequence and cDNA cloning.” Biochim Biophys Acta 1430(2): 262-268.
Shen, X., G. Ye, X. Cheng, C. Yu, H. Yao and C. Hu (2010). “Novel antimicrobial peptides identified from an endoparasitic wasp cDNA library.” J Pept Sci 16(1): 58-64.
Steiner, H., D. Hultmark, A. Engstrom, H. Bennich and H. G. Boman (1981). “Sequence and specificity of two antibacterial proteins involved in insect immunity.” Nature 292(5820): 246-248.
Tashiro, M., R. Tejero, D. E. Zimmerman, B. Celda, B. Nilsson and G. T. Montelione (1997). “High-resolution solution NMR structure of the Z domain of staphylococcal protein A.” J Mol Biol 272(4): 573-590.
Tsien, R. Y. (1998). “The green fluorescent protein.” Annu Rev Biochem 67: 509-544.
Waldo, G. S., B. M. Standish, J. Berendzen and T. C. Terwilliger (1999). “Rapid protein-folding assay using green fluorescent protein.” Nat Biotechnol 17(7): 691-695.
Xu, T., E. Telser, R. F. Troxler and F. G. Oppenheim (1990). “Primary Structure and Anticandidal Activity of the Major Histatin from Parotid Secretion of the Subhuman Primate, Macaca fascicularis.” Journal of Dental Research 69(11): 1717-1723.
Zhao, H. L., X. Q. Yao, C. Xue, Y. Wang, X. H. Xiong and Z. M. Liu (2008). “Increasing the homogeneity, stability and activity of human serum albumin and interferon-alpha2b fusion protein by linker engineering.” Protein Expr Purif 61(1): 73-77.
Zhao, H. L., C. Xue, J. L. Du, M. Ren, S. Xia and Z. M. Liu (2012). “Balancing the pharmacokinetics and pharmacodynamics of interferon-alpha2b and human serum albumin fusion protein by proteolytic or reductive cleavage increases its in vivo therapeutic efficacy.” Mol Pharm 9(3): 664-670.

1-32. (canceled)

33. A bi- or multifunctional fusion protein and/or fusion peptide comprising:
one or more of a first adhesion promoting protein and/or adhesion promoting peptide (I), which binds to one or more of a first polymer or plastic target surface;
(ii) one or more of a second adhesion promoting protein and/or adhesion promoting peptide (II), which binds to one or more of a second polymer or non-polymer carrier surface;
(iii) optionally, a spacer unit between the first and the second adhesion promoting protein and/or adhesion promoting peptide, whereby the first and the second adhesion promoting protein and/or adhesion promoting peptide are bonded together by the spacer unit, and
(iv) optionally, one or more of a function for generating one or more of a signal.

34. The bi- or multifunctional fusion protein and/or fusion peptide of claim 33, wherein the one or more first polymer or plastic target surface is selected from the group consisting of polyolefin, polyethylene (PE), polypropylene (PP), polyurethane (PU), polystyrene (PS), polyvinyl chloride (PVC), polycarbonate (PC), polyamide (PA), polyoxymethylene (POM), polymethyl methacrylate (PMMA), polyethylene terephthalate (PET), polybutylene terephthalate (PBT), polytetrafluoroethylene (PTFE), polyhydroxyalkanoate (PHA), polyhydroxybutyrate (PHB), polyimide (PI), polylactide (PLA), polyvinylidene fluoride (PVDF) and polyetherketone (PEK etc.), polyamides (PA), and/or polymeric or plastic foams, and copolymers or coplastics thereof.

35. The bi- or multifunctional fusion protein and/or fusion peptide of claim 33, wherein the one or more first polymer or plastic target surface is selected from the group consisting of polyethylene (PE), polypropylene (PP), polystyrene (PS), polyurethane (PU), and/or polymeric or plastic foams, and copolymers or coplastics thereof.

36. The bi- or multifunctional fusion protein and/or fusion peptide of claim 33, wherein the one or more first adhesion promoting protein and/or adhesion promoting peptide (I), and/or the one or more second adhesion promoting protein and/or adhesion promoting peptide (II), independently from each other, is selected from the group consisting of anchor peptides I and/or II having 2 to 180 amino acids.

37. The bi- or multifunctional fusion protein and/or fusion peptide of claim 33, wherein the one or more first adhesion promoting protein and/or adhesion promoting peptide (I), and/or the one or more second adhesion promoting protein and/or adhesion promoting peptide (II), independently from each other, is selected from peptides having 2 to 180 amino acids which have ability to integrate into membranes of microorganisms.

38. The bi- or multifunctional fusion protein and/or fusion peptide of claim 33, wherein the one or more first adhesion promoting protein and/or adhesion promoting peptide (I) is selected from the group consisting of Cecropin A, Tachystatin A2 (TA2), Thanatin (THA), Liquid Chromaography Peak 1 (LCI), Androctonin (ANR), Dermaseptin S1 (DS1), hDermcidin (hDerm), and a combination thereof.

39. The bi- or multifunctional fusion protein and/or fusion peptide of claim 33, wherein the one or more first adhesion promoting protein and/or adhesion promoting peptide (I) is an anchor peptide I selected from the group consisting of Tachystatin A2 (TA2), Thanatin (THA), Liquid Chromaography Peak 1 (LCI), Dermaseptin S1 (DS1), and a combination thereof.

40. The bi- or multifunctional fusion protein and/or fusion peptide of claim 33, wherein the one or more second adhesion promoting protein and/or adhesion promoting peptide (II) is an anchor peptide II selected from the group consisting of Tachystatin A2 (TA2), Thanatin (THA), Liquid Chromaography Peak 1 (LCI), Dermaseptin S1 (DS1), hDermcidin (hDerm), and a combination thereof.

41. The bi- or multifunctional fusion protein and/or fusion peptide according to claim 40, wherein the one or more second adhesion promoting protein and/or adhesion promoting peptide (II) binds to one or more of a second polymer and/or non-polymer carrier surface.

42. The bi- or multifunctional fusion protein and/or fusion peptide according to claim 41, wherein the one or more second polymer and/or non-polymer carrier surface is selected from the group consisting of metallic, metalized, ceramic, ceramized, glass, glassy, enamel, enamelled materials, woven materials, fiber materials, membrane materials, and a combination thereof; preferably a metallic or metalized material, silver (Ag), titanium (Ti), Gold (Au), stainless steel, and/or a magnetic material.

43. The bi- or multifunctional fusion protein and/or fusion peptide according to claim 41, wherein the one or more second polymer and/or non-polymer carrier surface is selected from the group consisting of titanium (Ti), Gold (Au), and/or stainless steel.

44. The bi- or multifunctional fusion protein and/or fusion peptide according to claim 33 comprising spacer unit (iii), wherein the spacer unit is a separator protein.

45. The bi- or multifunctional fusion protein and/or fusion peptide according to claim 44, wherein the spacer unit is a domain Z separator protein.

46. A bi- or multifunctional fusion protein and/or fusion peptide according to claim 45, wherein the spacer unit is a separator protein of a Staphylococcal protein A domain Z.

47. A system comprising:
(A) the bi- or multifunctional fusion protein and/or fusion peptide of claim 33; and
(B) the one or more of second polymer or non-polymer carrier surface, which is bonded to the one or more second adhesion promoting protein and/or adhesion promoting peptide (II).

48. A kit comprising:
(A) the bi- or multifunctional fusion protein and/or fusion peptide of claim 33; and
(B) a second component comprising one or more of a second polymer or non-polymer carrier surface, which selectively binds to the one or more second adhesion promoting protein and/or adhesion promoting peptide (II).

49. A method of detecting one or more target polymers or target particles in an environment, the method comprising:
a) providing the bi- or multifunctional fusion protein and/or fusion peptide of claim 33;
b) providing a second polymer or non-polymer carrier;
c) providing a liquid medium comprising one or more target polymers or target plastics;
d) contacting the liquid medium of c) with the fusion protein and/or fusion peptide of a), and allowing the fusion protein and/or fusion peptide to bind to at least a part or all of the one or more target polymers or target plastics in the liquid medium of c) and bind with the second polymer or non-polymer carrier of b);
e) removing the liquid medium of c);
f) optionally, removing at least a part or all of the one or more target polymers or target plastics bound by the fusion protein and/or fusion peptide; and
g) detecting the one or more target polymers or target plastics bound to the fusion protein and/or fusion peptide.

50. A method of separating one or more target polymers or target plastics from an environment comprising:
a) providing the bi- or multifunctional fusion protein and/or fusion peptide of claim 33;
b) providing a second polymer or non-polymer carrier;
c) providing a liquid medium comprising one or more target polymers or target plastics;
d) contacting the liquid medium of c) with the fusion protein and/or fusion peptide of a), and allowing the fusion protein and/or fusion peptide to bind to at least a part or all of the one or more target polymers or target plastics in the liquid medium of c) and bind with the second polymer or non-polymer carrier of b);
e) removing the liquid medium of c);
f) optionally, removing at least a part or all of the one or more target polymers or target plastics bound by the fusion protein and/or fusion peptide; and
g) optionally, continuously or batch-wise repeating steps a) to e), and/or optionally steps a) to f).

51. A method of preparing a bi- or multifunctional fusion protein and/or fusion peptide of claim 33 comprising:
a) providing:
(i) one or more of a first adhesion promoting protein and/or adhesion promoting peptide (I), which binds to one or more of a first polymer or plastic target surface;
(ii) one or more of a second adhesion promoting protein and/or adhesion promoting peptide (II), which binds to one or more of a second polymer or non-polymer carrier surface;
(iii) a spacer unit between the first and the second adhesion promoting protein and/or adhesion promoting peptide, whereby the first and the second adhesion promoting protein and/or adhesion promoting peptide are bonded together by the spacer unit, and
(iv) optionally, one or more of a function for generating one or more of a signal;
b) providing a ligation method;
c) fusing of the one or more first adhesion promoting protein and/or adhesion promoting peptide provided in (i) and the one or more first adhesion promoting protein and/or adhesion promoting peptide provided in (ii), by the ligation method b);
d) carrying out fusion step c) such that a spacer unit provided in (iii) is fused in between the first, provided in (i), and the second, provided in (ii), adhesion promoting protein and/or adhesion promoting peptide, whereby the first provided in (i) and the second provided in (ii) adhesion promoting protein and/or adhesion promoting peptide are bonded together by the said spacer unit (iii); and
e) optionally, fusing one or more function (iv) for generating one or more signal, before or after carrying out any of fusion steps c) and/or d); and
f) collecting, and optionally purifying, the bi- or multifunctional fusion protein and/or fusion peptide.
